(19) United States
(12) Patent Application Publication
(10) Pub. No.: US 2018/0151807 A1
(43) Pub. Date: May 31, 2018

(54) ORGANIC COMPOSITION AND ELECTRONIC DEVICE COMPRISING ORGANIC LAYER COMPRISING SAID COMPOSITION

(71) Applicants: Rohm and Haas Electronic Materials Korea Ltd., Cheonan (KR); Dow Global Technologies LLC, Midland, MI (US)

(72) Inventors: Zhengming Tang, Shanghai (CN); Moo Keun Chee, Daejeon (KR); Hong Yeop Na, Hwaseong (KR); Hua Ren, Shanghai (CN); Shaoguang Feng, Shanghai (CN)

(21) Appl. No.: 15/576,292

(22) PCT Filed: May 29, 2015
(86) PCT No.: PCT/CN2015/080225

§ 371 Date: Nov. 22, 2017

(51) Int. Cl. H01L 51/00; C09K 11/06; C07D 417/14

(52) U.S. Cl. CPC H01L 51/0071; C09K 11/06; C07D 417/14; H01L 51/0067; H01L 51/5056; H01L 51/0072; H01L 51/0074; H01L 51/0073; C09K 2211/1018; H01L 51/0077

(57) ABSTRACT

A composition suitable for organic layers of electronic devices that shows improved luminescent properties, and electronic devices comprising organic layers comprising such composition.

### FIELD OF THE INVENTION

The present invention relates to an organic composition and an electronic device comprising an organic layer comprising the composition.

### INTRODUCTION

Organic light emitting diodes (OLEDs) are display devices that employ stacks of films containing organic aromatic compounds in electron transport layers (ETLs) and hole transport layers (HTLs). It is desirable to develop materials with improved luminescent properties such as reduced driving voltage and/or increased luminous efficiency to minimize power consumption in OLED displays, especially for mobile applications where batteries are used as power sources. There has been a tremendous amount of research to develop materials to reduce driving voltages and increase luminous efficiency, mostly for hole injection materials (HIMs), such as described in Synthetic Metals, 2009, 159, 69 and J. Phys. D: Appl. Phys. 2007, 40, 5553. For electron transport layers, traditionally used materials such as tris(8-hydroxyquinolinato)aluminum (Alq₃) usually provide unsatisfactory luminescent properties. Thus, there remains a need for new compounds suitable for preparing electron transport layers of OLEDs which have improved luminescent properties than those comprising Alq₃-based electron transport layers.

Therefore, it is desirable to provide new compounds that are suitable to be used as electron transport materials capable of providing improved luminescent properties.

### SUMMARY OF THE INVENTION

The present invention provides a novel composition comprising an organic compound, and an electronic device comprising an organic layer comprising the composition. The electronic device of the present invention shows better luminescent properties than devices comprising Alq₃ as an electron transport material.

In a first aspect, the present invention provides a composition comprising an organic compound, wherein the organic compound has the structure selected from one of the following formulae (I-1) through (I-7):

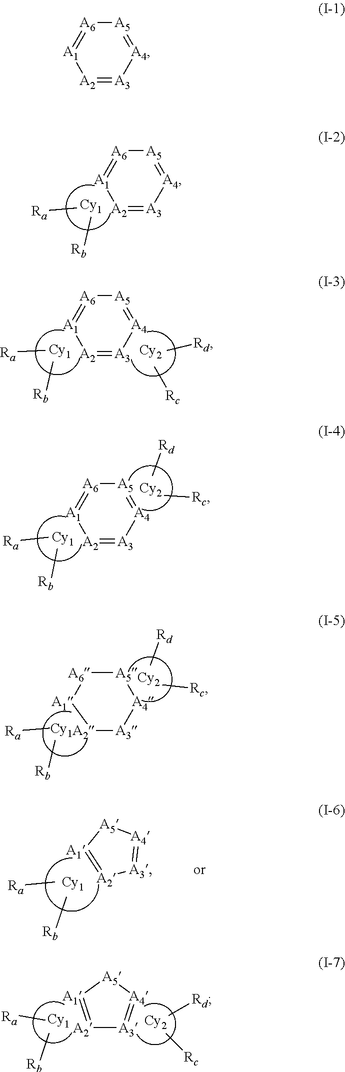

wherein, in formula (I-1), one or two of A₁ through A₆ are each independently selected from N, P, P═O, PR₁R₂ or B; and the remaining A₁ through A₆ are each independently selected from C or CR′; provided that at most one of A₁ through A₆ is N; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; and wherein two of A₁ through A₆ are substituted by Structure A;

wherein, in formula (I-2), A₁ and A₂ are each C; one or two of A₃ through A₆ are each independently selected from N, P, P═O, PR₁R₂ or B; and the remaining A₃ through A₆ are each independently selected from C or CR′; provided that at most one of A₃ through A₆ is N; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ is a five-membered or six-membered ring containing at most two heteroatoms, provided that at most one of the heteroatoms is N; wherein Rₐ and R_{b} are each independently selected from hydrogen, deuterium, a C₁-C₅₀ substituted or unsubstituted alkyl, a substituted or unsubstituted C₁-C₅₀ alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and Rₐ and R_{b} may optionally form a ring; and wherein two of A₃ through A₆ and atoms in Cy₁ are substituted by Structure A;

wherein, in formula (I-3), A₁ through A₄ are each C; one of A₅ and A₆ is selected from N, P, P═O, PR₁R₂ or B; and the remaining A₅ or A₆ is selected from C or CR′; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ and Cy₂ are each independently a five-membered or six-membered ring containing at most two heteroatoms, provided that at most one of the heteroatoms is N; wherein Rₐ, R_{b}, R_{c} and R_{d} are each independently selected from hydrogen, deuterium, a C₁-C₅₀ substituted or unsubstituted alkyl, a substituted or unsubstituted C₁-C₅₀ alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and Rₐ and R_{b}, or R_{c} and R_{d} may optionally form a ring; and wherein two of A₅ through A₆ and atoms in Cy₁ and Cy₂ are substituted by Structure A;

wherein, in formula (I-4), A₁, A₂, A₄ and A₅ are each C; one of A₃ and A₆ is selected from N, P, P═O, PR₁R₂ or B; and the remaining A₃ or A₆ is selected from C or CR′; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ and Cy₂ are each independently a five-membered or six-membered ring containing at most two heteroatoms, provided that at most one of the heteroatoms is N; wherein Rₐ, R_{b}, R_{c} and R_{d} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a substituted or unsubstituted C₆-C₅₀ aryloxy, a substituted or unsubstituted C₆-C₆₀ arylthio, a C₁-C₆₀ substituted or unsubstituted heteroaryl, a halogen or a cyano; and Rₐ and R_{b}, or R_{c} and R_{d} may optionally form a ring; and wherein two of A₃, A₆ and atoms in Cy₁ and Cy₂ are substituted by Structure A;

wherein, in formula (I-5), A₁″, A₂″, A₄″ and A₅″ are each C; and A₃″ and A₆″ are each independently selected from NR′, O, S or CR₁R₂; provided that at least one of A₃″ and A₆″ is selected from NR′, O or S; and at most one of A₃″ and A₆″ is NR′; wherein R′, R₁, and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ and Cy₂ are each independently a five-membered or six-membered ring containing at most two heteroatoms, provided that at most one of the heteroatoms is N; wherein Rₐ, R_{b}, R_{c} and R_{d} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and Rₐ and R_{b} may optionally form a ring; and wherein two of atoms in Cy₁ and Cy₂ are substituted by Structure A;

wherein, in formula (I-6), A₁′ and A₂′ are each C; A₃′ and A₄′ are each independently selected from C, CR′, N, P, P═O, PR₁R₂ or B; and A₅′ is selected from O, S, NR′ or CR₁R₂; provided that only one or two of A₁′ through A₅′ are not C, CR′ or CR₁R₂; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ is a five-membered or six-membered ring containing at most two heteroatoms, provided that at most one of the hertoatoms is N; wherein Rₐ and R_{b} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and Rₐ and R_{b} may optionally form a ring; and wherein two of A₃ through A₄ and atoms in Cy₁ are substituted by Structure A;

wherein, in formula (I-7), A₁′ through A₄′ are each C; and A₅′ is selected from O, S or NR′; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ and Cy₂ are each independently a five-membered or six-membered ring containing at most two heteroatoms, provided that at most one of the heteroatoms is N; wherein Rₐ, R_{b}, R_{c} and R_{d} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and Rₐ and R_{b}, or R_{c} and R_{d} may optionally form a ring; and wherein two of atoms in Cy₁ and Cy₂ are substituted by Structure A; and

wherein Structure A has the following structure:

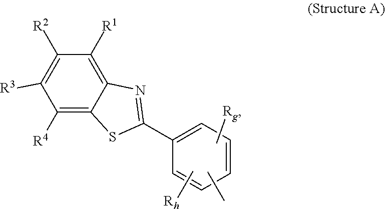

wherein R¹ through R⁴, R_{g} and Rₕ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a substituted or unsubstituted C₆-C₆₀ aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and R_{g} and Rₕ may optionally form a ring.

In a second aspect, the present invention provides an electronic device comprising an organic layer, wherein the organic layer comprises the composition of the first aspect.

### DETAILED DESCRIPTION OF THE INVENTION

The term “electronic device” refers to a device which depends on the principles of electronics and uses the manipulation of electron flow for its operation.

The term “light emitting device” herein refers to a device that emits light when an electrical current is applied across two electrodes.

The term “emitting layer” means a layer which consists of host and dopant. The host material could be bipolar or unipolar, and may be used alone or by combination of two or more host materials. The opto-electrical properties of the host material may differ to which type of dopant (phosphorescent or fluorescent) is used. For fluorescent dopants, the assisting host materials should have good spectral overlap between adsorption of the dopant and emission of the host to induce good Foester transfer to dopants. For phosphorescent dopants, the assisting host materials should have high triplet energy to confine triplets of the dopant.

The term “hole transport layer (HTL)” refers to a layer made from a material, which transports holes. High hole mobility is recommended for OLED devices. The HTL is used to help block passage of electrons transported by the emitting layer. Small electron affinity is typically required to block electrons. The HTL should desirably have larger triplets to block exciton migrations from an adjacent EML layer. Examples of HTL compounds include, but are not limited to, di(p-tolyl)aminophenyl]cyclohexane (TPAC), N,N-diphenyl-N,N-bis(3-methylphenyl)-1,1-biphenyl-4,4-diamine (TPD), and N,N′-diphenyl-N,N′-bis(1-naphthyl)-(1,1′-biphenyl)-4,4′-diamine (NPB).

The term “dopant” refers to an electron acceptor or a donator that increases the conductivity of an organic layer of an organic electronic device, when added to the organic layer as an additive. Organic semiconductors may likewise be influenced, with regard to their electrical conductivity, by doping. Such organic semiconducting matrix materials may be made up either of compounds with electron-donor properties or of compounds with electron-acceptor properties.

The term “heteroatoms” include O, N, P, P(═O), Si, B and S.

The term “unsubstituted aryl” refers to an organic radical derived from aromatic hydrocarbon by the removal of one hydrogen atom therefrom. An aryl group may be a monocyclic and/or fused ring system each ring of which suitably contains from 4 to 6, preferably from 5 or 6 atoms. Structures wherein two or more unsubstituted aryl groups are combined through single bond(s) are also included.

The term “substituted aryl” refers to an aryl in which at least one hydrogen atom is substituted with a heteroatom or a chemical group containing at least one heteroatom.

Heteroatoms may include, for example, O, N, P and S. The chemical group containing at least one heteroatom herein may include, for example, OR′, NR′₂, PR′₂, P(═O)R′₂, SiR′₃; where each R′ is a C₁-C₃₀ hydrocarbyl group.

The term “hydrocarbyl” refers to a chemical group containing only hydrogen and carbon atoms.

The term “unsubstituted heteroaryl” refers to an aryl group, in which at least one carbon atom or CH group or CH₂ group is substituted with a heteroatom (for example, B, N, O, S, P(═O), Si and P) or a chemical group containing at least one heteroatom. The unsubstituted heteroaryl may be a 5- or 6-membered monocyclic heteroaryl or a polycyclic heteroaryl which is fused with one or more benzene ring(s), and may be partially saturated. The structures having one or more unsubstituted heteroaryl group(s) bonded through a single bond are also included. The unsubstituted heteroaryl groups may include divalent aryl groups of which the heteroatoms are oxidized or quarternized to form N-oxides, quaternary salts, or the like.

The term “substituted heteroaryl” refers to a heteroaryl in which at least one hydrogen atom is substituted with a heteroatom or a chemical group containing at least one heteroatom. Heteroatoms may include, for example, O, N, P and S. The chemical group containing at least one heteroatom may include, for example, OR′, NR′₂, PR′₂, P(═O)R′₂, or SiR′₃, wherein each R′ is a C₁-C₃₀ hydrocarbyl group.

The term “alkyl” refers a saturated hydrocarbon group and other substituents containing “alkyl” moiety include both linear and branched species. Examples of alkyls include methyl, ethyl, propyl, iso-propyl, butyl, iso-butyl, tert-butyl, pentyl, or hexyl.

The term “substituted alkyl” refers to a saturated hydrocarbon group having a linear and branched structure in which at least one hydrogen atom is substituted with a heteroatom or a chemical group containing at least one heteroatom. Heteroatoms may include, for example, O, N, P and S. The chemical group containing at least one heteroatom herein may include, for example, OR′, NR′₂, PR′₂, P(═O)R′₂, or SiR′₃; where each R′ is a C₁-C₃₀ hydrocarbyl group.

In the present invention, a substituted group, unless otherwise stated, refers to a group containing one or more Substituent B. Substituent B may include, for example, deuterium, halogen, C₁-C₃₀ alkyl with or without halogen substituent(s), C₆-C₃₀ aryl, C₁-C₃₀ heteroaryl with or without C₆-C₃₀ aryl substituent(s), a 5- to 7-membered heterocycloalkyl containing one or more heteroatom(s) selected from, for example, B, N, O, S, P(═O), Si and P, a 5 to 7-membered heterocycloalkyl fused with one or more aromatic ring(s), C₃-C₃₀ cycloalkyl, C₅-C₃₀ cycloalkyl fused with one or more aromatic ring(s), tri(C₁-C₃₀) alkylsilyl, di(C₁-C₃₀)alkyl(C₆-C₃₀)arylsilyl, tri(C₆-C₃₀)arylsilyl, adamantyl, C₇-C₃₀ bicycloalkyl, C₂-C₃₀ alkenyl, C₂-C₃₀ alkynyl, cyano, carbazolyl; BR₆R₇, PR₈R₉, or P(═O)R₁₀R₁₁, wherein R₆ through R₁₁ independently represent C₁-C₃₀ alkyl, C₆-C₃₀ aryl or C₁-C₃₀ heteroaryl; C₁-C₃₀ alkyloxy, C₁-C₃₀ alkylthio, C₆-C₃₀ aryloxy, C₆-C₃₀ arylthio, C₁-C₃₀ alkoxycarbonyl, C₁-C₃₀ alkylcarbonyl, C₆-C₃₀ arylcarbonyl, C₆-C₃₀ aryloxycarbonyl, C₁-C₃₀ alkoxycarbonyloxy, C₁-C₃₀ alkylcarbonyloxy, C₆-C₃₀ arylcarbonyloxy, C₆-C₃₀ aryloxycarbonyloxy, carboxyl, nitro and hydroxyl; or that the substituents are linked together to form a ring. For example, a substituent may form a ring structure with one or more atoms on the backbone molecule comprising said substituent.

The term “cycloalkyl” includes a monocyclic hydrocarbon and a polycyclic hydrocarbon such as substituted or unsubstituted adamantyl or substituted or unsubstituted C₇-C₃₀ bicycloalkyl.

The composition of the present invention comprises one or more organic compounds.

In one embodiment, the organic compound in the composition has the structure represented by formula (I-1):

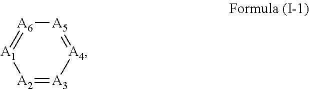

wherein, in formula (I-1), one or two of A₁ through A₆ are each independently selected from N, P, P═O, PR₁R₂, or B; and the remaining A₁ through A₆ are each independently CR′; provided that at most one of A₁ through A₆ is N;

wherein, in formula (I-1), R′, R₁, and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; and

wherein, in formula (I-1), two of A₁ through A₆ are substituted by Structure A having the following structure:

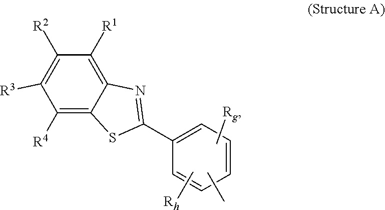

wherein, in Structure A, R¹ through R⁴, R_{g} and Rₕ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen, or a cyano; and R_{g} and Rₕ may optionally form a ring. That is, the organic compound of Formula (I) comprises two Structure A substituents. The two Structure A substituents may be the same or different. Preferably, R¹ through R⁴, R_{g} and Rₕ are each independently selected from hydrogen, a C₆-C₆₀ substituted or unsubstituted aryl, or a C₁-C₆₀ substituted or unsubstituted heteroaryl. More preferably, R¹ through R⁴, R_{g} and Rₕ are each independently selected from hydrogen.

In one preferred embodiment, in formula (I-1), one of A₁ through A₆ is N and the remaining A₁ through A₆ are each independently CR′. In more preferred embodiments, in formula (I-1), R¹ through R⁴, R_{g} and Rₕ are each independently selected from hydrogen, a C₆-C₆₀ substituted or unsubstituted aryl, or a C₁-C₆₀ substituted or unsubstituted heteroaryl, and more preferably hydrogen; and one of A₁ through A₆ is N and the remaining A₁ through A₆ are each independently CR′.

In one embodiment, the organic compound in the composition of the present invention has the structure represented by formula (I-2):

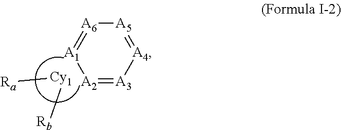

wherein, in formula (I-2), A₁ and A₂ are each C; one or two of A₃ through A₆ are each independently selected from N, P, P═O, PR₁R₂ or B; and the remaining A₃ through A₆ are each independently selected from C or CR′; provided that at most one of A₃ through A₆ is N;

wherein, in formula (I-2), R′, R₁, and R₂ are each as previously described in formula (I-1);

wherein, in formula (I-2), Rₐ and R_{b} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen, or a cyano; and Rₐ and R_{b} may optionally form a ring;

wherein, in formula (I-2), Cy₁ is a five-membered or six-membered ring containing at most two heteroatoms, provided that at most one of the heteroatoms is N; and

wherein, in formula (I-2), two of A₃ through A₆ and atoms in Cy₁ are substituted by Structure A described above. That is, the organic compound of formula (I-2) contains two Structure A substituents. The two Structure A substituents may be the same or different.

In one embodiment, in formula (I-2), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably hydrogen. In another embodiment, A₁ and A₂ are each C, one of A₃ through A₆ is N, and the remaining A₃ through A₆ are each independently selected from C or CR′.

In more preferred embodiments, in formula (I-2), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably hydrogen; and A₁ and A₂ are each C, one of A₃ through A₆ is N, and the remaining A₃ through A₆ are each independently selected from C or CR′.

In some preferred embodiments, the organic compound of formula (I-2) has the structure represented by formula (II-2a) or (II-2b):

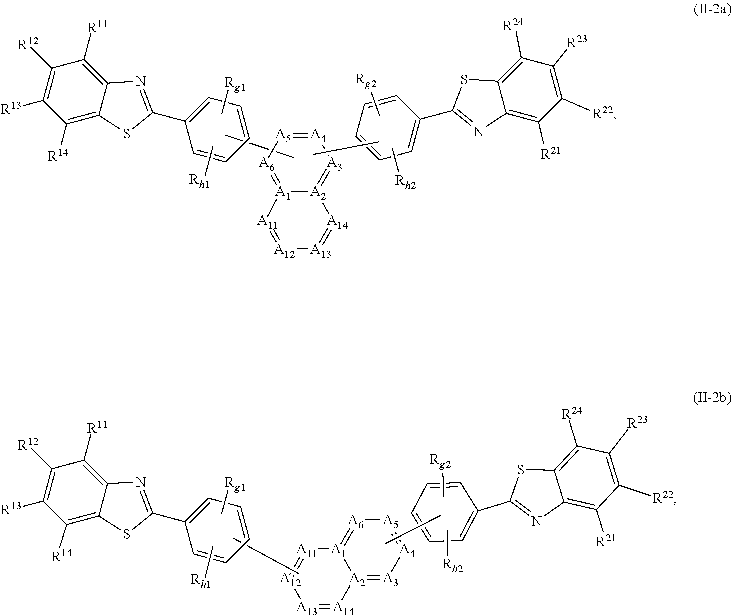

wherein, in each of formula (II-2a) and (II-2b), A₁ and A₂ are C; and A₃ through A₆ and A₁₁ through A₁₄ are each independently selected from CR′, N, P, P═O, PR₁R₂, or B; provided that one of A₃ through A₆ is N and at most one of A₁₁ through A₁₄ is N;

wherein, in each of formula (II-2a) and (II-2b), R′, R₁, and R₂ are each as previously described in formula (I-1); and

wherein, in each of formula (II-2a) and (II-2b), R¹¹ through R¹⁴, R²¹ through R²⁴, R_{g1}, Rₕ₁, R_{g1} and Rₕ₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen, or a cyano; and R_{g1} and Rₕ₁, R_{g2} and Rₕ₂ may optionally form a ring, and two adjacent of R¹¹ through R¹⁴, or R²¹ through R²⁴ may optionally form a ring.

In each of formula (II-2a) and (II-2b), the ring constituted by A₁ through A₆ and the ring constituted by A₁, A₂, and A₁₁ through A₁₄ may each independently contain no more than 2 heteroatoms. Examples of

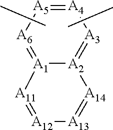

in formula (II-2a) or

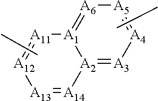

in formula (II-2b) include:

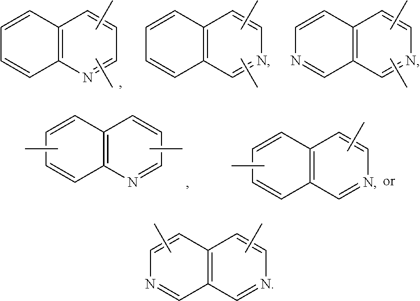

In one preferred embodiment, in each of formula (II-2a) and (II-2b), R¹¹ through R¹⁴, R²¹ through R²⁴, R_{g1}, Rₕ₁, R_{g2} and Rₕ₂ are each independently selected from hydrogen, a C₆-C₆₀ substituted or unsubstituted aryl, or a C₁-C₆₀ substituted or unsubstituted heteroaryl. More preferably, R¹¹ through R¹⁴, R²¹ through R²⁴, R_{g1}, Rₕ₁, R_{g2} and Rₕ₂ are each hydrogen.

In one embodiment, the organic compound in the composition of the present invention has the structure represented by formula (I-3):

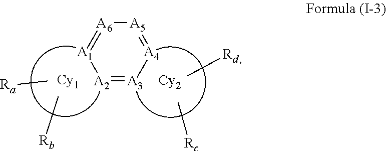

wherein, in formula (I-3), A₁ through A₄ are each C; one of A₅ and A₆ is selected from N, P, P═O, PR₁R₂ or B; and the remaining A₅ and A₆ is C or CR′;

wherein, in formula (I-3), R′, R₁, and R₂ are each as previously described in formula (I-1);

wherein, in formula (I-3), Cy₁ and Cy₂ are each independently a five-membered or six-membered ring containing at most two heteroatoms, provided that at most one of the herteroatoms is N;

wherein, in formula (I-3), Rₐ, R_{b}, R_{c}, and R_{d} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen, or a cyano; and Rₐ and R_{b}, or R_{c} and R_{d} may optionally form a ring; and

wherein, in formula (I-3), two of A₅ through A₆ and atoms in Cy₁ and Cy₂ are substituted by Structure A described above. That is, the organic compound of formula (I-3) contains two Structure A substituents. The two Structure A substituents may be the same or different.

In one embodiment, in formula (I-3), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably, R¹ through R⁴, R_{g} and Rₕ are each hydrogen. In another embodiment, A₁ through A₄ are each C, one of A₅ and A₆ is selected from N, and the remaining A₅ or A₆ is selected from C or CR′.

In more preferred embodiments, in formula (I-3), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably, R¹ through R⁴, R_{g} and Rₕ are each hydrogen; and A₁ through A₄ are each C, one of A₅ and A₆ is selected from N; and the remaining A₅ or A₆ is selected from C or CR′.

In some preferred embodiments, the organic compound of formula (I-3) has the structure represented by formula (II-3a) or (II-3b):

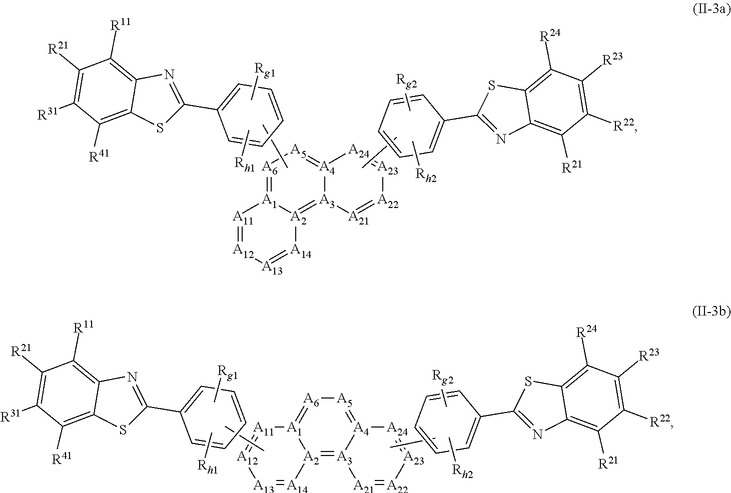

wherein, in each of formula (II-3a) and (II-3b), A₁ through A₄ are each C; one of A₅ and A₆ is selected from N, P, P═O, PR₁R₂ or B; and the remaining A₅ or A₆ is selected from C or CR′; and A₁₁ through A₁₄ and A₂₁ through A₂₄ are each independently selected from C, CR′, N, P, P═O, PR₁R₂ or B; provided that at most one of A₁₁ through A₁₄ is N, and at most one of A₂₁ through A₂₄ is N;

wherein, in each of formula (II-3a) and (II-3b), R′, R₁, and R₂ are as previously described in formula (I-1); and

wherein, in each of formula (II-3a) and (II-3b), R¹¹ through R¹⁴, R²¹ through R²⁴, R_{g1}, Rₕ₁, R_{g1} and Rₕ₂ are each as previously described in formula (II-2a) or (II-2b).

In one preferred embodiment, in each of formula (II-3a) and (II-3b), R¹¹ through R¹⁴, R²¹ through R²⁴, R_{g1}, Rₕ₁, R_{g2}, and Rₕ₂ are each independently selected from hydrogen, a C₆-C₆₀ substituted or unsubstituted aryl, or a C₁-C₆₀ substituted or unsubstituted heteroaryl. More preferably, R¹¹ through R¹⁴, R²¹ through R²⁴, R_{g1}, Rₕ₁, R_{g2}, and Rₕ₂ are each hydrogen.

In another preferred embodiment, in formula (II-3a) and (II-3b), the ring constituted by A₁ through A₆, the ring constituted by A₁, A₂, and A₁ through A₁₄, and the ring constituted by A₃, A₄, and A₂₁ through A₂₄ each independently contains no more than 2 heteroatoms.

In one embodiment, the organic compound in the composition of the present invention has the structure represented by formula (I-4):

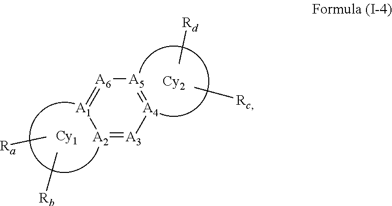

wherein, in formula (I-4), A₁, A₂, A₄ and A₅ are each C; one of A₃ and A₆ is selected from N, P, P═O, PR₁R₂ or B; and the remaining A₃ or A₆ is selected from C or CR′;

wherein, in formula (I-4), R′, R₁, and R₂ are as previously described in formula (I-1);

wherein, in formula (I-4), Cy₁ and Cy₂ are each independently a five-membered or six-membered ring containing at most two heteroatoms, provided that at most one of the herteroatoms is N;

wherein, in formula (I-4), Rₐ, R_{b}, R_{c}, and R_{d} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen, or a cyano; and Rₐ and R_{b}, or R_{c} and R_{d} may optionally form a ring; and

wherein, in formula (I-4), two of A₃, A₆ and atoms in Cy₁ and Cy₂ are substituted by Structure A described above. That is, the organic compound of formula (I-4) contains two Structure A substituents. The Structure A substituents may be the same or different.

In one embodiment, in formula (I-4), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably hydrogen. In another embodiment, A₁, A₂, A₄ and A₅ are each C; one of A₃ and A₆ is N; and the remaining A₃ or A₆ is selected from C or CR′.

In more preferred embodiments, in formula (I-4), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably hydrogen; and A₁, A₂, A₄ and A₅ are each C; one of A₃ and A₆ is N; and the remaining A₃ or A₆ is selected from C or CR′.

In some preferred embodiments, the organic compound of formula (I-4) has the structure represented by formula (II-4):

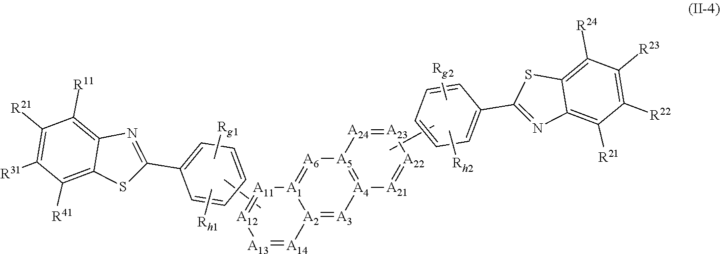

wherein, in formula (II-4), A₁, A₂, A₄ and A₅ are each C; one of A₃ and A₆ is selected from N, P, P═O, PR₁R₂ or B; and the remaining A₃ or A₆ is selected from C or CR′; and A₁₁ through A₁₄, and A₂₁ through A₂₄ are each independently selected from C, CR′, N, P, P═O, PR₁R₂ or B; provided that at most one of A₁₁ through A₁₄ is N, and at most one of A₂₁ through A₂₄ is N;

wherein, in formula (II-4), R′, R₁, and R₂ are as previously described in formula (I-1); and

wherein, in formula (II-4), R¹¹ through R¹⁴, R²¹ through R²⁴, R_{g1}, Rₕ₁, R_{g1} and Rₕ₂ are each as previously described in formula (II-2a) or (II-2b).

In one preferred embodiment, in formula (II-4), R¹¹ through R¹⁴, R²¹ through R²⁴, R_{g1}, Rₕ₁, R_{g2}, and Rₕ₂ are each independently selected from hydrogen, a C₆-C₆₀ substituted or unsubstituted aryl, or a C₁-C₆₀ substituted or unsubstituted heteroaryl. More preferably, R¹¹ through R¹⁴, R²¹ through R²⁴, R_{g1}, Rₕ₁, R_{g2}, and Rₕ₂ are each hydrogen.

Examples of

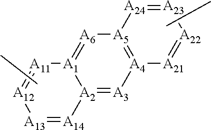

in formula (II-4) include

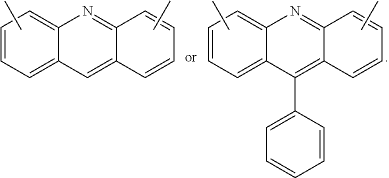

In one embodiment, the organic compound in the composition of the present invention has the structure represented by formula (I-5):

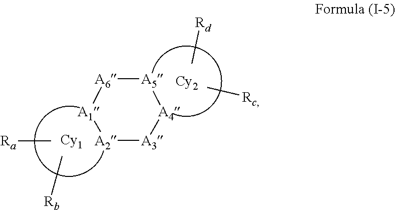

wherein, in formula (I-5), A₁″, A₂″, A₄″ and A₅″ are each C; and A₃″ and A₆″ are each independently selected from NR′, O, S or CR₁R₂; provided that at least one of A₃″ and A₆″ is selected from NR′, O or S; and at most one of A₃″ and A₆″ is NR′;

wherein, in formula (I-5), R′, R₁, and R₂ are as previously defined in formula (I-1);

wherein, in formula (I-5), Cy₁ and Cy₂ are each independently a five-membered or six-membered ring containing at most two heteroatoms, provided that at most one of the heteroatoms is N;

wherein, in formula (I-5), Rₐ, R_{b}, R_{c} and R_{d} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstitutedaryl, a C₆-C₅₀ substituted or unsubstituted aryloxy, a C₆-C₆₀ substituted or unsubstituted arylthio, a C₁-C₆₀ substituted or unsubstituted heteroaryl, a halogen, or a cyano; and Rₐ and R_{b} may optionally form a ring; and

wherein, in formula (I-5), two of atoms in Cy₁ and Cy₂ are substituted by Structure A described above. That is, the organic compound of formula (I-5) contains two Structure A substituents. The two Structure A substituents may be the same or different.

In one embodiment, in formula (I-5), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably hydrogen. In another preferred embodiment, at least one of A₃″ and A₆″ is NR′.

In more preferred embodiments, in formula (I-5), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably hydrogen; and at least one of A₃″ and A₆″ is NR′.

In some preferred embodiments, the organic compound of formula (I-5) has the structure represented by formula (II-5):

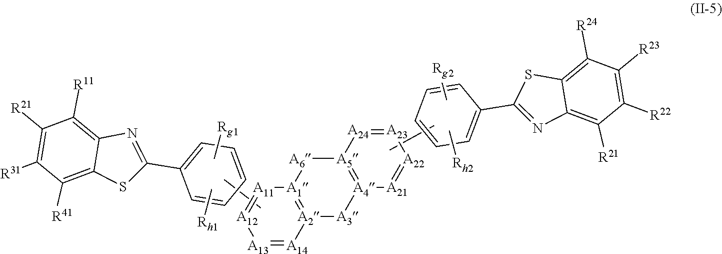

wherein A₁″, A₂″, A₄″ and A₅″ are each C; A₃″ and A₆″ are each independently selected from NR′, O, S, or CR₁R₂; A₁₁ through A₁₄ and A₂₁ through A₂₄ are each independently selected from C, CR′, N, P, P═O, PR₁R₂ or B; provided that one of A₃″ and A₆″ is NR′, at most one of A₁₁ through A₁₄ is N, and at most one of A₂₁ through A₂₄ is N;

wherein R′, R₁, and R₂ are as previously defined in formula (I-1); and

wherein R¹¹ through R¹⁴, R²¹ through R²⁴, R_{g1}, Rₕ₁, R_{g1} and Rₕ₂ are each as previously described in formula (II-2a) or (II-2b).

In one preferred embodiment, in formula (II-5), R¹¹ through R¹⁴, R²¹ through R²⁴, R_{g1}, Rₕ₁, R_{g2}, and Rₕ₂ are each independently selected from hydrogen, a C₆-C₆₀ substituted or unsubstituted aryl, or a C₁-C₆₀ substituted or unsubstituted heteroaryl. More preferably, R¹¹ through R¹⁴, R²¹ through R²⁴, R_{g1}, Rₕ₁, R_{g2}, and Rₕ₂ are each hydrogen.

Examples of

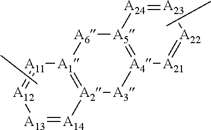

in formula (II-5) include

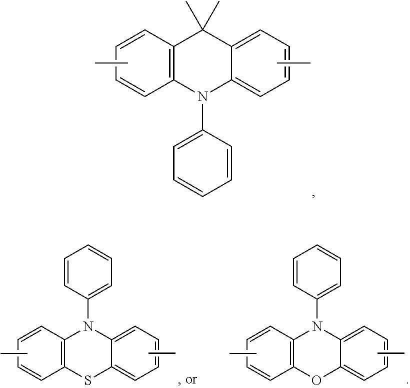

In one embodiment, the organic compound in the composition of the present invention has the structure represented by formula (I-6):

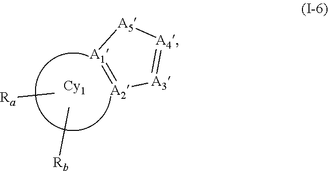

wherein, in formula (I-6), A₁′ and A₂′ are each C; A₃′ and A₄′ are each independently selected from C, CR′, N, P, P═O, PR₁R₂ or B; and A₅′ is selected from O, S, NR′ or CR₁R₂; provided that only one or two of A₁′ through A₅′ are not C, CR′ or CR₁R₂;

wherein, in formula (I-6), R′, R₁, and R₂ are as previously described in formula (I-1);

wherein, in formula (I-6), Cy₁ is a five-membered or six-membered ring containing at most two heteroatoms, provided that at most one of the heteroatoms is N;

wherein, in formula (I-6), Rₐ and R_{b} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen, or a cyano; and Rₐ and R_{b} may optionally form a ring; and

wherein, in formula (I-6), two of A₃ through A₄ and atoms in Cy₁ are substituted by Structure A described above. That is, the organic compound of formula (I-6) contains two Structure A substituents. The two Structure A substituents may be the same or different.

In one embodiment, in formula (I-6), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably hydrogen. In another embodiment, A₁′ through A₄′ are each independently selected from C or CR′, and A₅′ is selected from O, S, NR′ or CR₁R₂.

In more preferred embodiments, in formula (I-6), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably hydrogen; and A₁′ through A₄′ are each independently selected from C or CR′, and A₅′ is selected from O, S, NR′ or CR₁R₂.

In some preferred embodiments, the organic compound of formula (6) has the structure represented by formula (II-6a) or (II-6b):

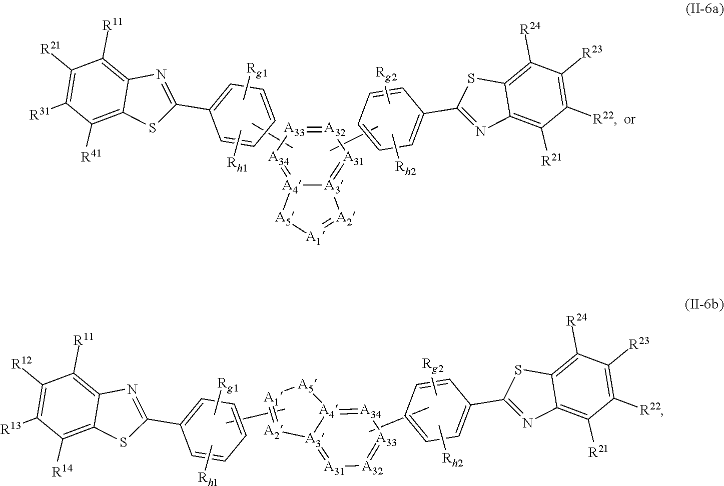

wherein, in each of formula (II-6a) and (II-6b), A₃′ and A₄′ are each C; A₁′ and A₂′ are each independently selected from C, CR′, N, P, P═O, PR₁R₂ or B; A₅′ is selected from O, S, NR′ or CR₁R₂; and A₃₁ through A₃₄ are each independently selected from C, CR′, N, P, P═O, PR₁R₂, or B; provided that only one or two of A₁′, A₂′ and A₅′ are not C, CR′ or CR₁R₂; and at most one of A₃₁ through A₃₃ is N;

wherein, in formula (II-6a) and (II-6b), R′, R₁, and R₂ are as previously described in formula (I-1); and

wherein, in formula (II-6a) and (II-6b), R¹¹ through R¹⁴, R²¹ through R²⁴, R_{g1}, Rₕ₁, R_{g1} and Rₕ₂ are each as previously described in formula (II-2a) or (II-2b).

In one preferred embodiment, in formula (II-6a) and (II-6b), R¹¹ through R¹⁴, R²¹ through R²⁴, R_{g1}, Rₕ₁, R_{g2}, and Rₕ₂ are each independently selected from hydrogen, a C₆-C₆₀ substituted or unsubstituted aryl, or a C₁-C₆₀ substituted or unsubstituted heteroaryl. More preferably, R¹¹ through R¹⁴, R²¹ through R²⁴, R_{g1}, Rₕ₁, R_{g2}, and Rₕ₂ are each hydrogen.

Examples of

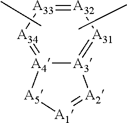

in formula (II-6a) or

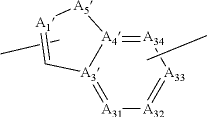

in formula (II-6b)
include,

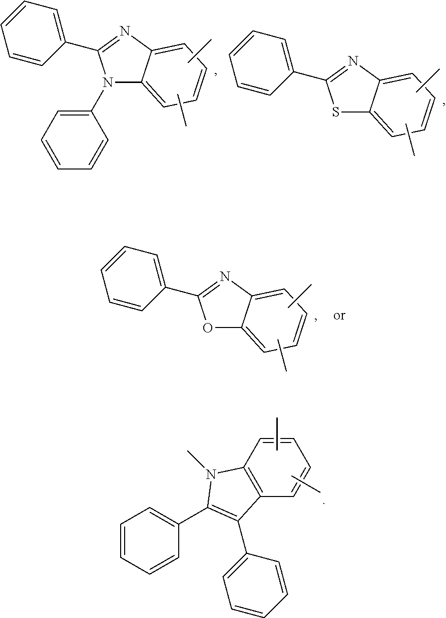

In one embodiment, the organic compound in the composition of the present invention has the structure represented by formula (I-7):

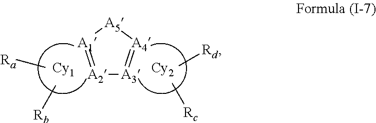

wherein, in formula (I-7), A₁′ through A₄′ are each independently selected from C, CR′, N, P, P═O, PR₁R₂ or B; and A₅′ is selected from O, S, NR′ or CR₁R₂; provided that only one or two of A₁′ through A₅′ are not C, CR′ or CR₁R₂;

wherein, in formula (I-7), R′, R₁, and R₂ are as previously described in formula (I-1);

wherein, in formula (I-7), Cy₁ and Cy₂ are each independently a five-membered or six-membered ring containing at most two heteroatoms, provided that at most one of the heteroatoms is N;

wherein, in formula (I-7), Rₐ, R_{b}, R_{c}, and R_{d} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a substituted or unsubstituted C₆-C₆₀ aryl, a C₆-C₅₀ substituted or unsubstituted aryloxy, a C₆-C₆₀ substituted or unsubstituted arylthio, or a C₁-C₆₀ substituted or unsubstituted heteroaryl, a halogen, or a cyano; and Rₐ and R_{b}, or R_{c} and R_{d} may optionally form a ring; and

wherein, in formula (I-7), two of atoms in Cy₁ and Cy₂ are substituted by Structure Adescribed above. That is, the organic compound of formula (I-7) contains two Structure A substituents. The two Structure A substituents may be the same or different.

In one embodiment, in formula (I-7), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably hydrogen. In another embodiment, A₁′ through A₄′ are each independently selected from C or CR′, and A₅′ is selected from O, S or CR₁R₂.

In more preferred embodiments, in formula (I-7), R¹ through R⁴, R_{g} and Rₕ are each independently selected from hydrogen, a C₆-C₆₀ substituted or unsubstituted aryl, or a C₁-C₆₀ substituted or unsubstituted heteroaryl; and more preferably hydrogen; and A₁′ through A₄′ are each independently selected from C or CR′, and A₅′ is selected from O, S or CR₁R₂.

In some preferred embodiments, the organic compound of formula (I-7) has the structure represented by formula (II-7):

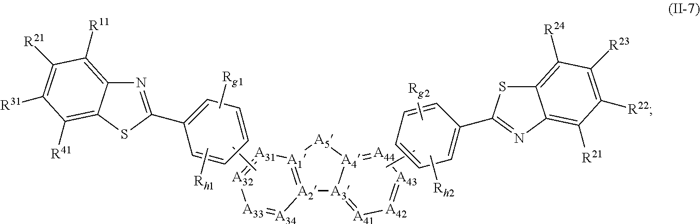

wherein A₁′ through A₄′ are each C; A₅′ is selected from O, S, or NR′; and A₃₁ through A₃₄ and A₄₁ through A₄₄ are each independently selected from C, CR′, N, P, P═O, PR₁R₂ or B; provided that at most one of A₃₁ through A₃₃ is N, and at most one of A₄₁ through A₄₄ is N;

wherein R′, R₁, and R₂ are as previously described in formula (I-1); and

wherein R¹¹ through R¹⁴, R²¹ through R²⁴, R_{g1}, Rₕ₁, R_{g1} and Rₕ₂ are each as previously described in formula (II-2a) or (II-2b).

In one preferred embodiment, in formula (II-7), R¹¹ through R¹⁴, R²¹ through R²⁴, R_{g1}, Rₕ₁, R_{g2}, and Rₕ₂ are each independently selected from hydrogen, a C₆-C₆₀ substituted or unsubstituted aryl, or a C₁-C₆₀ substituted or unsubstituted heteroaryl. More preferably, R¹¹ through R¹⁴, R²¹ through R²⁴, R_{g1}, Rₕ₁, R_{g2}, and Rₕ₂ are each hydrogen. Examples of

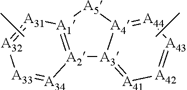

in formula (II-7) include:

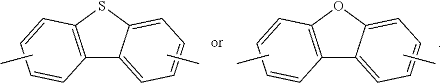

In each of Formula (I-1) through (I-7), (II-2a), (II-2b), (II-3a), (II-3b), (II-4), (II-5), (II-6a), (II-6b) and (II-7) described above, “C₆-C₆₀ substituted or unsubstituted aryl” can be C₆-C₅₀ substituted or unsubstituted aryl in one embodiment, C₆-C₃₀ substituted or unsubstituted aryl in another embodiment, C₆-C₂₀ substituted or unsubstituted aryl in still another embodiment, or C₆-C₁₂ substituted or unsubstituted aryl in yet another embodiment. Examples of the unsubstituted aryls include phenyl, naphthyl, biphenyl, anthryl, indenyl, fluorenyl, benzofluorenyl, phenanthryl, triphenylenyl, pyrenyl, perylenyl, chrysenyl, naphtacenyl, fluoranthenyl and the like. The naphthyl may be 1-naphthyl or 2-naphthyl. The anthryl may be 1-anthryl, 2-anthryl or 9-anthryl. The fluorenyl may be any one of 1-fluorenyl, 2-fluorenyl, 3-fluorenyl, 4-fluorenyl and 9-fluorenyl.

In each of Formula (I-1) through (I-7), (II-2a), (II-2b), (II-3a), (II-3b), (II-4), (II-5), (II-6a), (II-6b) and (II-7) described above, “C₆-C₅₀ substituted or unsubstituted aryloxy” can be C₆-C₃₀ substituted or unsubstituted aryloxy in one embodiment, C₆-C₂₀ substituted or unsubstituted aryloxy in another embodiment, or C₆-C₁₂ substituted or unsubstituted aryloxy in still another embodiment.

In each of Formula (I-1) through (I-7), (II-2a), (II-2b), (II-3a), (II-3b), (II-4), (II-5), (II-6a), (II-6b) and (II-7) described above, “C₆-C₆₀ substituted or unsubstituted arylthio” can be C₆-C₃₀ substituted or unsubstituted arylthio in one embodiment, C₆-C₂₀ substituted or unsubstituted arylthio in another embodiment, or C₆-C₁₂ substituted or unsubstituted arylthio in still another embodiment.

In each of Formula (I-1) through (I-7), (II-2a), (II-2b), (II-3a), (II-3b), (II-4), (II-5), (II-6a), (II-6b) and (II-7) described above, “C₁-C₆₀ substituted or unsubstituted heteroaryl” can be C₁-C₃₀ substituted or unsubstituted heteroaryl in one embodiment, C₂-C₂₀ substituted or unsubstituted heteroaryl in another embodiment, or C₄-C₁₂ substituted or unsubstituted heteroaryl in still another embodiment. Specific examples include, for example, monocyclic heteroaryl groups, such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl; polycyclic heteroaryl groups, such as benzofuranyl, fluoreno[4, 3-b]benzofuranyl, benzothiophenyl, fluoreno[4, 3-b]benzothiophenyl, isobenzofuranyl, benzimidazolyl, benzothiazolyl, benzisothiazolyl, benzisoxazolyl, benzoxazolyl, isoindolyl, indolyl, indazolyl, benzothia-diazolyl, quinolyl, isoquinolyl, cinnolinyl, quinazolinyl, quinoxalinyl, carbazolyl, phenanthridinyl and benzodioxolyl; and corresponding N-oxides (for example, pyridyl N-oxide, quinolyl N-oxide) and quaternary salts thereof.

In each of Formula (I-1) through (I-7), (II-2a), (II-2b), (II-3a), (II-3b), (II-4), (II-5), (II-6a), (II-6b) and (II-7) described above, “C₁-C₅₀ substituted or unsubstituted alkyl” can be C₁-C₃₀ substituted or unsubstituted alkyl in one embodiment, C₁-C₂₀ substituted or unsubstituted alkyl in another embodiment, C₁-C₁₀ substituted or unsubstituted alkyl in still another embodiment, C₁-C₅ substituted or unsubstituted alkyl in yet another embodiment, or C₁-C₃ substituted or unsubstituted alkyl in still yet another embodiment.

In each of Formula (I-1) through (I-7), (II-2a), (II-2b), (II-3a), (II-3b), (II-4), (II-5), (II-6a), (II-6b) and (II-7) described above, “C₁-C₅₀ substituted or unsubstituted alkoxy” can be C₁-C₃₀ substituted or unsubstituted alkoxy in one embodiment, C₁-C₂₀ substituted or unsubstituted alkoxy in another embodiment, C₁-C₁₀ substituted or unsubstituted alkoxy in still another embodiment, C₁-C₅ substituted or unsubstituted alkoxy in yet another embodiment, or C₁-C₃ substituted or unsubstituted alkoxy in still yet another embodiment.

In each of Formula (I-1) through (I-7), (II-2a), (II-2b), (II-3a), (II-3b), (II-4), (II-5), (II-6a), (II-6b) and (II-7) described above, “C₁-C₅₀ substituted or unsubstituted alkoxycarbonyl” can be C₁-C₃₀ substituted or unsubstituted alkoxycarbonyl in one embodiment, C₁-C₂₀ substituted or unsubstituted alkoxycarbonyl in another embodiment, C₁-C₁₀ substituted or unsubstituted alkoxycarbonyl in still another embodiment, C₁-C₅ substituted or unsubstituted alkoxycarbonyl in yet another embodiment, or C₁-C₃ substituted or unsubstituted alkoxycarbonyl in still yet another embodiment.

The organic compound in the composition of the present invention may be selected from the following compounds (1) through (27):

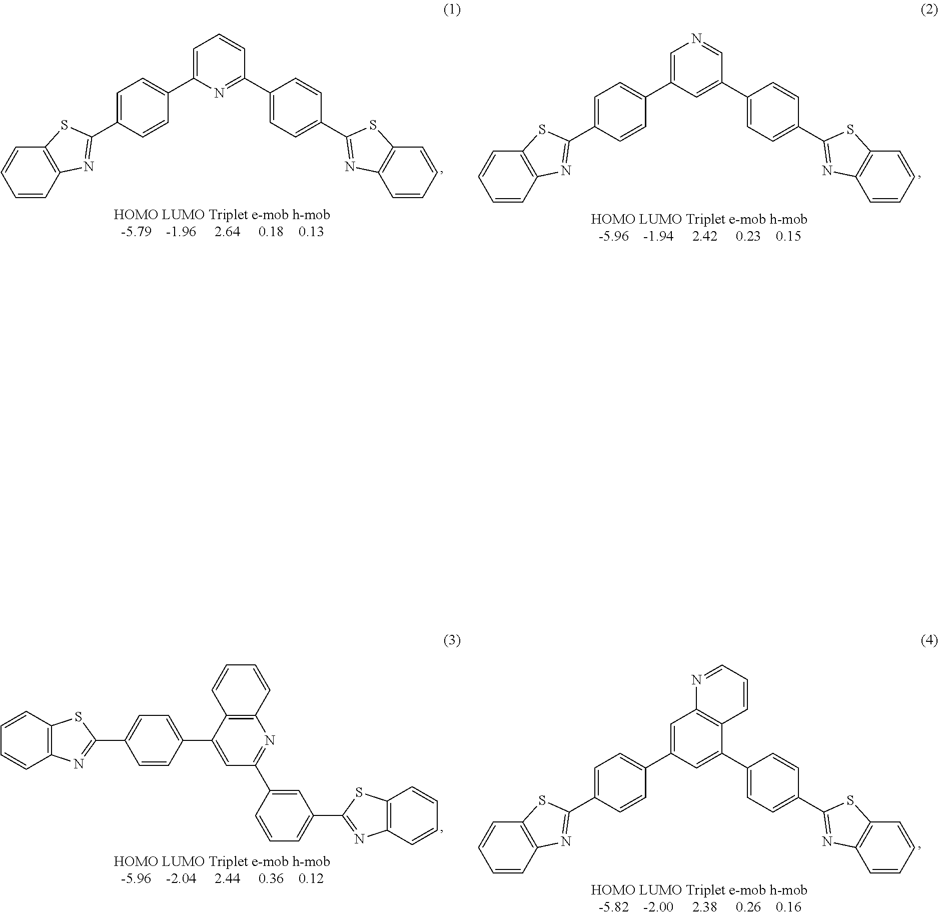 [SMILES: c1cc(-c2ccc(-c3nc4ccccc4s3)cc2)nc(-c2ccc(-c3nc4ccccc4s3)cc2)c1 ; c1ccc2sc(-c3ccc(-c4cncc(-c5ccc(-c6nc7ccccc7s6)cc5)c4)cc3)nc2c1 ; c1cc(-c2cc(-c3ccc(-c4nc5ccccc5s4)cc3)c3ccccc3n2)cc(-c2nc3ccccc3s2)c1 ; c1ccc2sc(-c3ccc(-c4cc(-c5ccc(-c6nc7ccccc7s6)cc5)c5cccnc5c4)cc3)nc2c1]

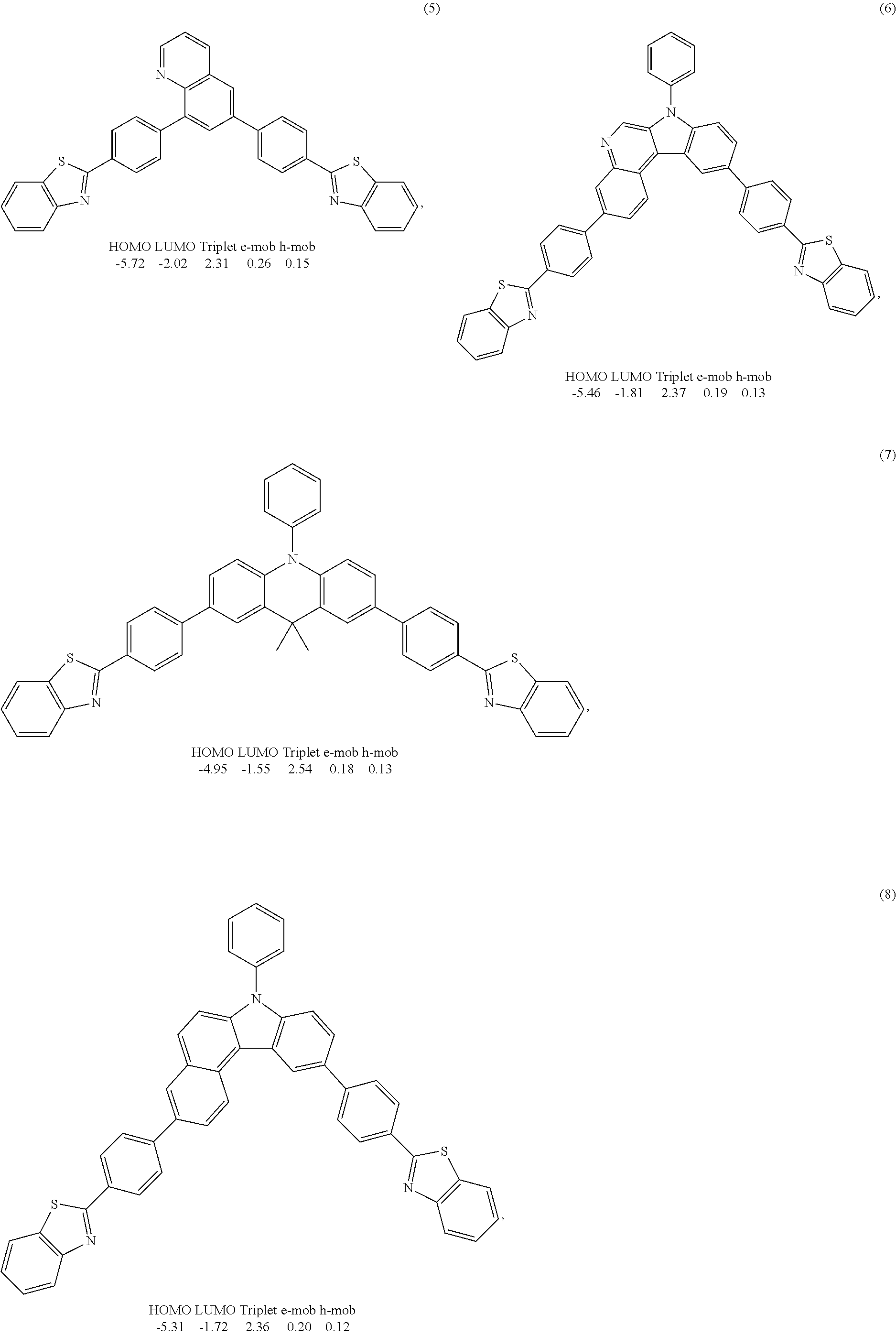 [SMILES: c1cnc2c(-c3ccc(-c4nc5ccccc5s4)cc3)cc(-c3ccc(-c4nc5ccccc5s4)cc3)cc2c1 ; c1ccc(-n2c3ccc(-c4ccc(-c5nc6ccccc6s5)cc4)cc3c3c4ccc(-c5ccc(-c6nc7ccccc7s6)cc5)cc4ncc32)cc1 ; CC1(C)c2cc(-c3ccc(-c4nc5ccccc5s4)cc3)ccc2N(c2ccccc2)c2ccc(-c3ccc(-c4nc5ccccc5s4)cc3)cc21 ; c1ccc(-n2c3ccc(-c4ccc(-c5nc6ccccc6s5)cc4)cc3c3c4ccc(-c5ccc(-c6nc7ccccc7s6)cc5)cc4ccc32)cc1]

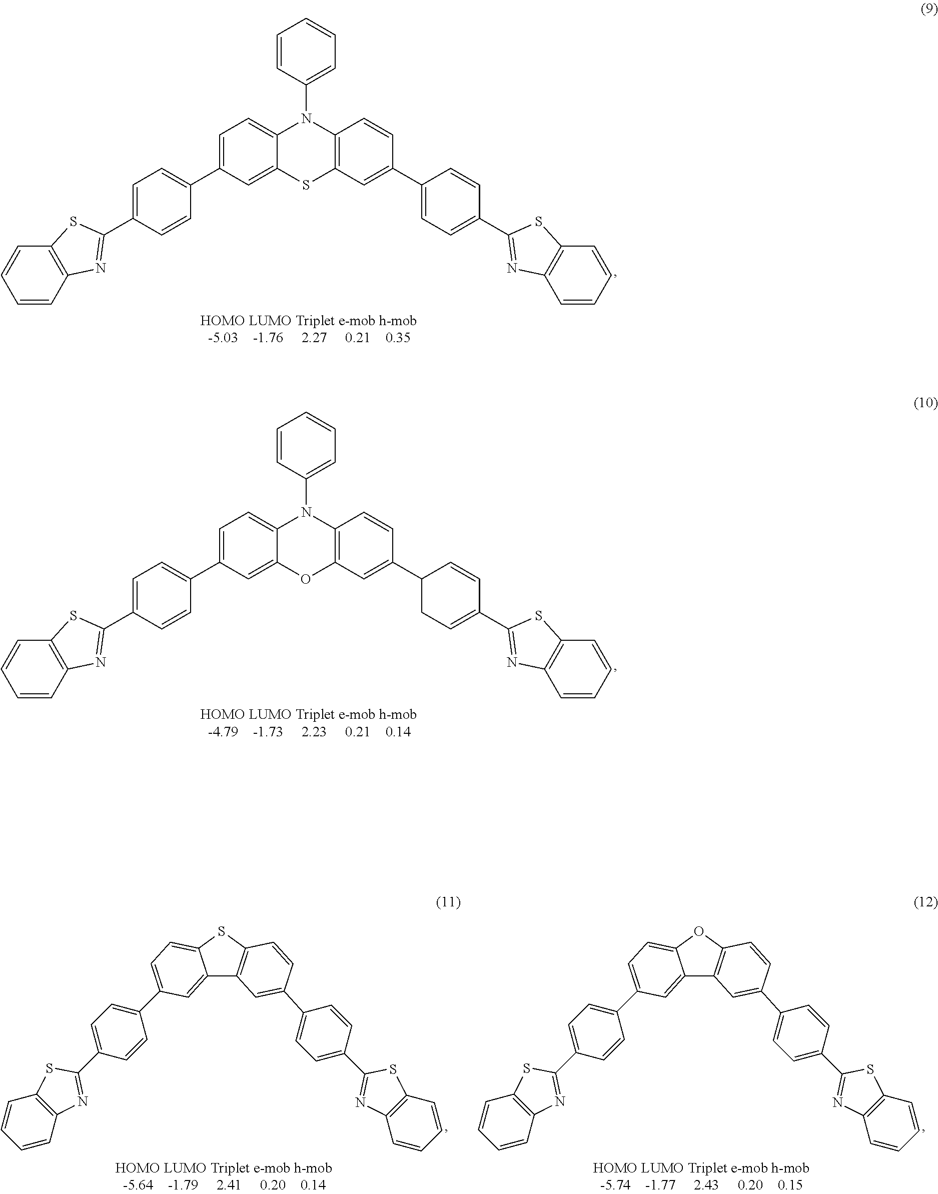

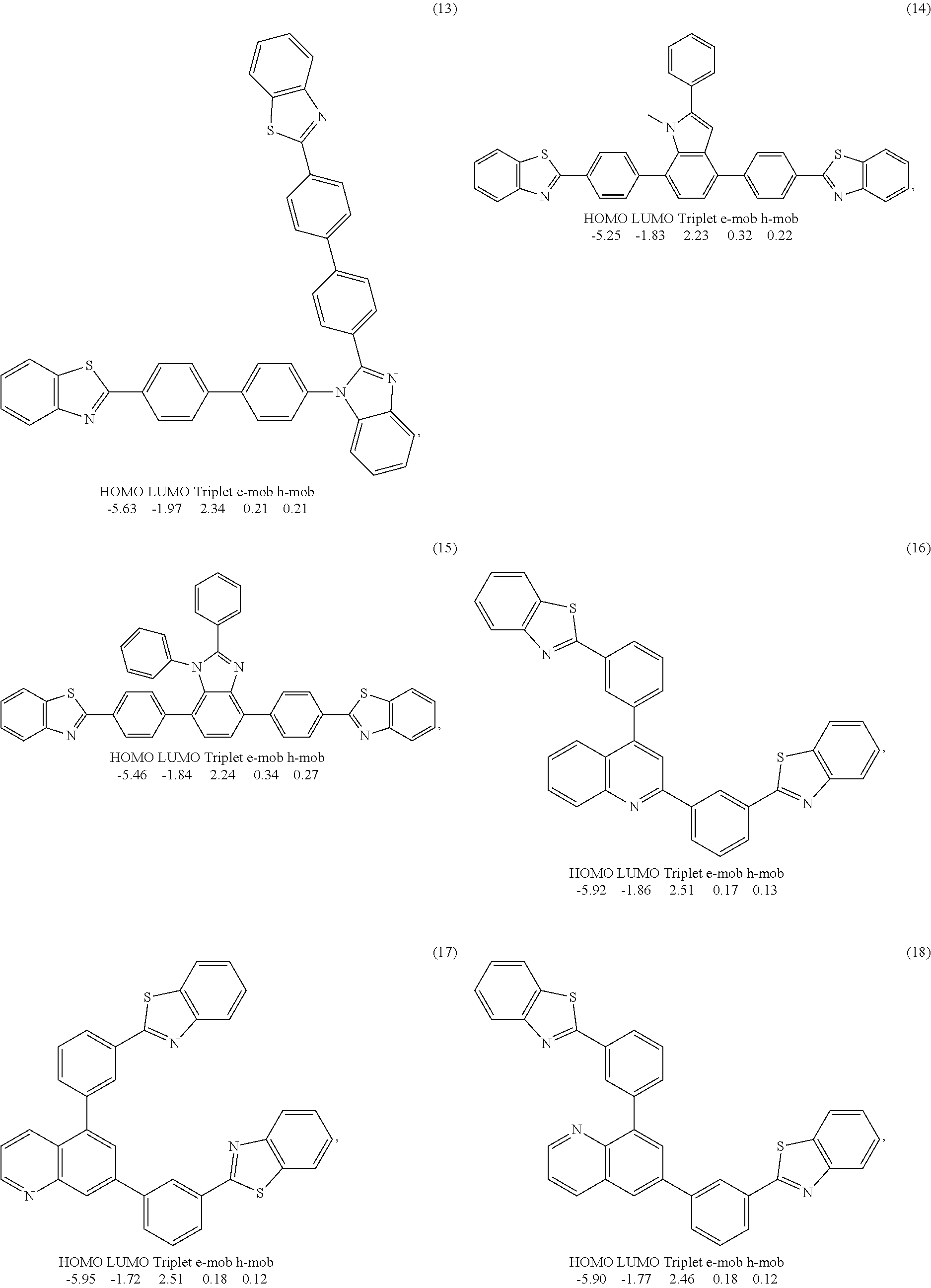 [SMILES: c1ccc2sc(-c3ccc(-c4ccc(-c5nc6ccccc6n5-c5ccc(-c6ccc(-c7nc8ccccc8s7)cc6)cc5)cc4)cc3)nc2c1 ; Cn1c(-c2ccccc2)cc2c(-c3ccc(-c4nc5ccccc5s4)cc3)ccc(-c3ccc(-c4nc5ccccc5s4)cc3)c21 ; c1ccc(-c2nc3c(-c4ccc(-c5nc6ccccc6s5)cc4)ccc(-c4ccc(-c5nc6ccccc6s5)cc4)c3n2-c2ccccc2)cc1 ; c1cc(-c2cc(-c3cccc(-c4nc5ccccc5s4)c3)c3ccccc3n2)cc(-c2nc3ccccc3s2)c1 ; c1cc(-c2cc(-c3cccc(-c4nc5ccccc5s4)c3)c3cccnc3c2)cc(-c2nc3ccccc3s2)c1 ; c1cc(-c2cc(-c3cccc(-c4nc5ccccc5s4)c3)c3ncccc3c2)cc(-c2nc3ccccc3s2)c1]

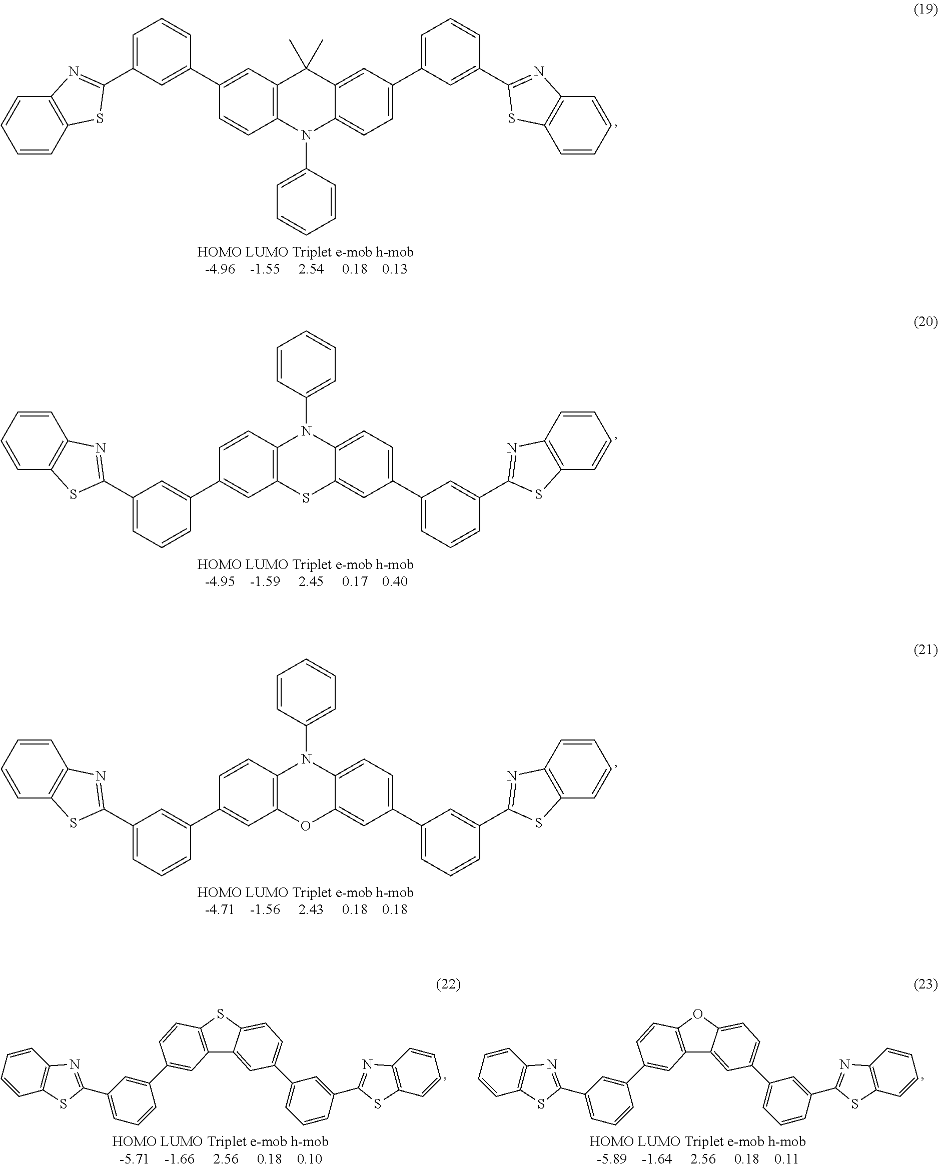

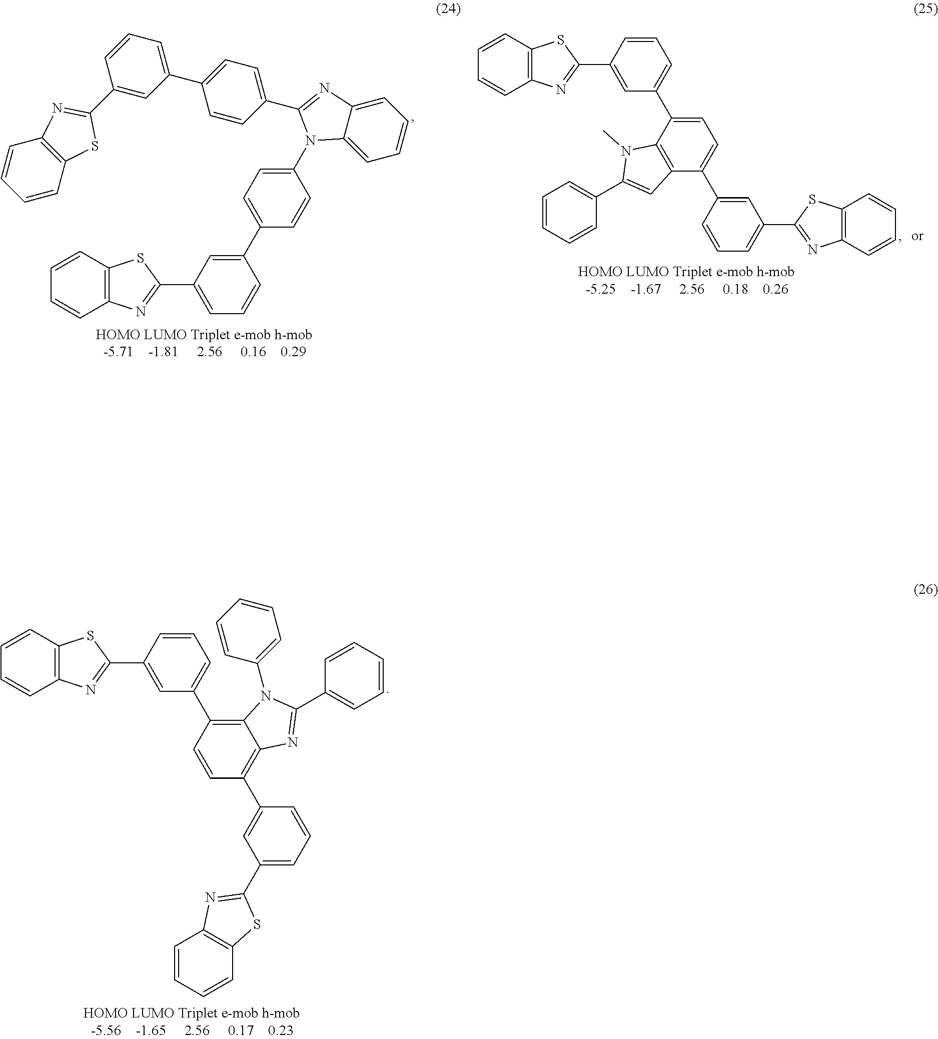 [SMILES: c1cc(-c2ccc(-c3nc4ccccc4n3-c3ccc(-c4cccc(-c5nc6ccccc6s5)c4)cc3)cc2)cc(-c2nc3ccccc3s2)c1 ; Cn1c(-c2ccccc2)cc2c(-c3cccc(-c4nc5ccccc5s4)c3)ccc(-c3cccc(-c4nc5ccccc5s4)c3)c21 ; c1ccc(-c2nc3c(-c4cccc(-c5nc6ccccc6s5)c4)ccc(-c4cccc(-c5nc6ccccc6s5)c4)c3n2-c2ccccc2)cc1]

The composition of the present invention may comprise a mixture of two or more of the organic compounds having the same or different formula as described above.

The organic compound in the composition of the present invention may have a molecular weight of 400 g/mole or more, 600 g/mole or more, or even 800 g/mole or more, and at the same time, 1200 g/mole or less, 1000 g/mole or less, or even 800 g/mole or less.

The organic compound in the composition of the present invention may have the highest occupied molecular orbital (HOMO) level from −4.0 to −7.0 electronvolts (eV) or from −5.0 to −6.0 eV. The organic compound in the composition of the present invention may have the lowest unoccupied molecular orbital (LUMO) level from −1.5 to −2.2 eV or from −1.6 to −2.0 eV. The organic compound in the composition of the present invention may have a triplet energy of from 1.5 to 3.5 eV or from 1.6 to 3.2 eV. The HOMO, LUMO, and triplet energy may be determined according to the test method described in the Examples section below.

The organic compound in the composition of the present invention may have a melting temperature (Tₘ) of 60° C. or higher, 80° C. or higher, or 100° C. or higher, and at the same time, 200° C. or lower, 180° C. or lower, or even 160° C. or lower.

The organic compound in the composition of the present invention may have a decomposition temperature (T_{d}) at 5% weight loss of 200° C. or higher, 250° C. or higher, or 300° C. or higher, and at the same time, 500° C. or lower, 480° C. or lower, or even 450° C. or lower, as measured according to the test method described in the Examples section below.

The organic compound in the composition of the present invention may be prepared by conventional methods in the art, for example, as shown in Scheme 1. A boric ester derivative of 2-phenylbenzo[d]thiazole may react with an aromatic compound with two halogen atoms (X) through a Suzuki coupling reaction to give the organic compound. Examples of suitable catalysts for the Suzuki reaction include, for example, Pd(PPh₃)₄, Pd(PPh₃)₂Cl₂, Pd(dppf)Cl₂, or mixtures thereof. The reaction can be conducted in the presence of one or more bases. Suitable bases include Na₂CO₃, K₂CO₃, K₃PO₄, NaOH or mixtures thereof. The reaction can also be conducted in the presence of one or more solvents. Suitable solvents include, for example, toluene, tetrahydrofuran (THF), xylene or mixtures thereof.

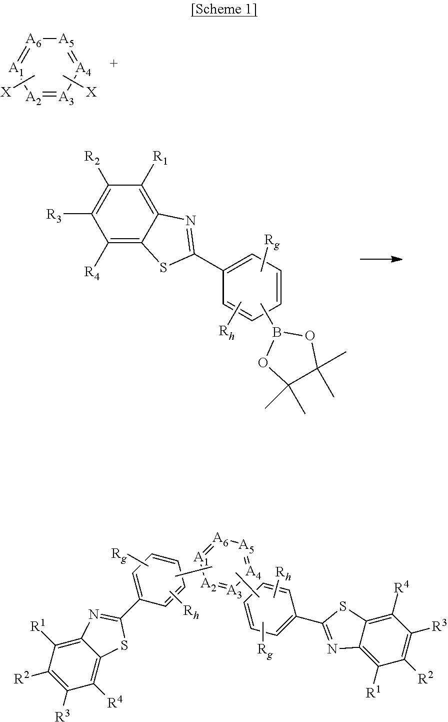

wherein A through A₆, R₁ through R₄, R_{g} and Rₕ are as previously defined with reference to Formula (I-1).

The composition of the present invention may further comprise or be free of one or more dopants. In one preferred embodiment, in addition to the organic compound described above, the organic layer comprises one or more dopants. Dopants may include different salts of 8-hydroxyquinoline. Examples of suitable dopants include

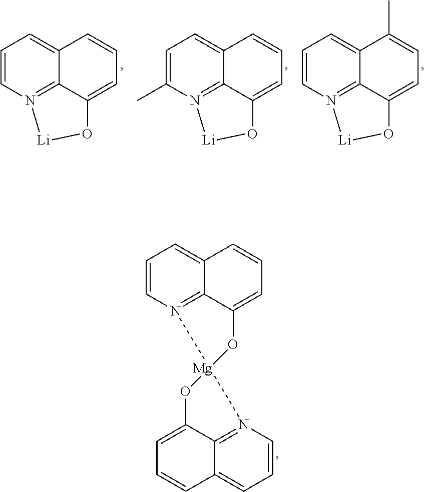

or mixtures thereof.

The concentration of the dopant may be, based on the total weight of the composition of the present invention, from 0 to 100% by weight, from 5% to 80% by weight, or from 10% to 70% by weight.

The composition of the present invention may be used as charge transport layers and other organic layers in electronic devices, such as OLED devices. For example, the organic compound of the present invention may be used as charge blocking layers and charge generation layers.

The present invention also provides a film. The film includes, or is otherwise composed of (formed from), the composition of the present invention. The film may be formed in an evaporative process or in a solution process.

The present invention also provides an electronic device comprising an organic layer comprising the organic composition of the present invention. The electronic device may include power generation; organic photovoltaics; organic sensors; organic memory devices; organic field effect transistors; and light emitting devices such as OLED devices; and storage devices such as organic batteries, fuel cells, and organic supercapacitors.

The electronic device of the present invention may comprise a first electrode; a second electrode; and one or more organic layers interposed between the first electrode and the second electrode, wherein the organic layer comprises the composition of the present invention. The organic layer can be a charge transfer layer that can transport charge carrying moieties, either holes or electrons. The organic layer may comprise a hole transport layer, an emissive layer, an electron transport layer, or a hole injection layer. The organic layer comprising the composition of the present invention may be prepared by evaporative vacuum deposition or a solution process such as spin coating and ink-jet printing.

### EXAMPLES

The following examples illustrate embodiments of the present invention. All parts and percentages are by weight unless otherwise indicated.

All solvents and reagents were obtained from commercial vendors, and were used in the highest available purities, and/or when necessary, recrystallized before use. Dry solvents were obtained from an in-house purification/dispensing system (hexane, toluene and THF), or purchased from Sigma-Aldrich. All experiments involving “water sensitive compounds” were conducted in “oven dried” glassware, under nitrogen (N₂) atmosphere, or in a glovebox. Reactions were monitored by analytical thin-layer chromatography (TLC) on precoated aluminum plates (VWR 60 F254), visualized by UV light and/or potassium permanganate staining. Flash chromatography was performed on an ISCO COMBIFLASH system with GRACERESOLV cartridges.

| Material | | | CAS |
|---|---|---|---|
| name | Chemical structure | Supplieer | No. |
| 2,6- dibromo- pyridine | 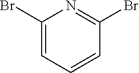 | Sinopharm Chemical Reagent Co., Ltd. (SCRC) | 626-05-1 |
| 3,5- dibromo- pyridine | 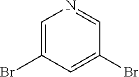 | SCRC | 625-92-3 |
| 4-bromo- benz- aldehyde | 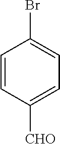 | SCRC | 1122-91-4 |
| 2-amino- benzene- thiol | 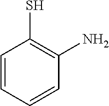 | SCRC | 137-07-5 |
| Bis (pinacolato) diboron | 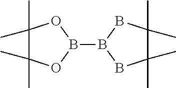 | Energy Chemical | 73183-34-3 |
| Tetrakis | Pd(PPh₃)₄ | Aldrich | 14221-01-3 |
| (triphenyl- | | | |
| phosphine) | | | |
| Palladium(0) | | | |
| [1,1′-Bis | Pd(dppf)Cl₂ | SCRC | 72287-26-4 |
| (diphenyl- | | | |
| phosphino) | | | |
| ferrocene] | | | |
| palladium(II) | | | |
| chloride | | | |

The following standard analytical equipment and methods are used in the Examples.

### Modeling

All computations utilized the Gaussian 09 program as described in Gaussian 09, Revision A.02, Frisch, M. J. et al., Gaussian, Inc., Wallingford Conn., 2009. The calculations were performed with the hybrid density functional theory (DFT) method, B3LYP as described in Becke, A. D. J. Chem. Phys. 1993, 98, 5648; Lee, C. et al., Phys. Rev B 1988, 37, 785; and Miehlich, B. et al., Chem. Phys. Lett. 1989, 157, 200; and the 6-31G* (5d) basis set as described in Ditchfield, R. et al., J. Chem. Phys. 1971, 54, 724; Hehre, W. J. et al., J. Chem. Phys. 1972, 56, 2257; and Gordon, M. S. Chem. Phys. Lett. 1980, 76, 163. The singlet state calculations use the closed shell approximation, and the triplet state calculations use the open shell approximation. All values are quoted in eV. The HOMO and LUMO values are determined from the orbital energies of the optimized geometry of the singlet ground state. The triplet energies are determined as the difference between the total energy of the optimized triplet state and the optimized singlet state. A procedure, as described in Lin, B. C. et al., J. Phys. Chem. A 2003, 107, 5241-5251, is applied to calculate the reorganization energy of each molecule, with which as the indicator of electron and hole mobility.

### Nuclear Magnetic Resonance (NMR)

¹H-NMR spectra (500 MHZ or 400 MHZ) are obtained on a Varian VNMRS-500 or VNMRS-400 spectrometer at 30° C. The chemical shifts are referenced to tetramethyl silane (TMS) (6:000) in CDCl₃.

### Differential Scanning Calorimetry (DSC)

DSC measurements are carried out on a TA Instruments Q2000 instrument at a scan rate of 10° C./min under N₂ atmosphere for all cycles. The sample (about 7-10 mg) is scanned from room temperature (20-25° C.) to 350° C., cooled to −60° C., and reheated to 350° C. T_{g} is measured on the second heating scan. Data analysis is performed using TA Universal Analysis software. The T_{g} value is calculated using an “onset-at-inflection” methodology.

### Thermal Gravimetric Analysis (TGA)

TGA measurements are carried out on a TA Instruments TGA-Q500 under N₂ atmosphere. The sample (about 7-10 mg) is weighed in a platinum standard plate and loaded into the instrument. The sample is first heated to 60.0° C. and equilibrated for 30 minutes to remove solvent residues in the sample. Then the sample is cooled to 30.0° C. The temperature is ramped from 30.0° C. to 600.0° C. with 10.0° C./min rate and the weight change is recorded to determine the decomposition temperature (T_{d}) of the sample. The temperature-weight % (T-Wt %) curve is obtained by TGA scan. The temperature at the 5% weight loss is determined as T_{d}.

### Liquid Chromatography-Mass Spectrometry (LC/MS)

A sample is dissolved in THF at around 0.6 mg/mL. 5 μL sample solution is injected on an Agilent 1220 HPLC/G6224A TOF mass spectrometer. The following analysis conditions are used:

Column: 4.6×150 mm, 3.5 μm ZORBAX Eclipse Plus C18; column temperature: 40° C.; Mobile phase: THF/deionized (DI) water=65/35 volume ratio (Isocratic method); Flow rate: 1.0 mL/min; and

MS conditions: Capillary Voltage: 3500 kV (Pos); Mode: Pos; Scan: 100-2000 amu; Rate: 1 s/scan; and Desolvation temperature: 300° C.

### High Performance Liquid Chromatography (HPLC)

A sample is dissolved in THF at around 0.6 mg/mL. The sample solution is at last filtrated through a 0.45 μm syringe filter and 5 μL of the filtrate is injected to HPLC system. The following analysis conditions are used:

Injection volume: 5 μL; Instrument: Agilent 1200 HPLC; Column: 4.6×150 mm, 3.5 μm ZORBAX Eclipse Plus C18; Column temperature: 40° C.; Detector: DAD=250, 280, 350 nm; Mobile Phase: THF/DI water=65/35 volume ratio (Isocratic method); and Flow rate: 1 mL/min.

### Synthesis of boric ester derivative of 2-phenylbenzo[d]thiazole

4-bromobenzaldehyde (2.01 g, 4.0 mmol, 185 g/mol), 2-aminobenzenethiol (1.25 g, 10.0 mmol, 125 g/mol) and acetic acid (Cat.) were charged in a 250 mL three-neck flask. The above mixture was heated at 70° C. and kept for 12 hours. TLC was utilized to monitor the reaction results. After cooling to room temperature, the resulting mixture was recrystalized in 20-30 mL alcohol and filtered to give white crystals. The obtained white crystals (2.90 g, 10.0 mmol, 290 g/mol), bis(pinacolato)diboron (2.54 g, 10.0 mmol, 254 g/mol), potassium acetate (2.45 g, 25.0 mmol, 98 g/mol), and Pd(dppf)Cl₂ (220 mg, 0.3 mmol, 732 g/mol) were then charged in a 250 mL three-neck flask. Dioxane (60 mL) was added into the above mixture under N₂ atmosphere and stirred at 80° C. for 6 hours. After cooling to room temperature, the resulting reaction mixture was extracted with ethyl acetate (EtOAc). The resulting extracts were washed with water and brine, dried over anhydrous Na₂SO₄, and filtered. Solvents were removed under reduced pressure and separated via silica gel column to give products as white solid powders with a yield of about 70% over two steps. The route of synthesis of the boric ester derivative of 2-phenylbenzo[d]thiazole is shown as follows:

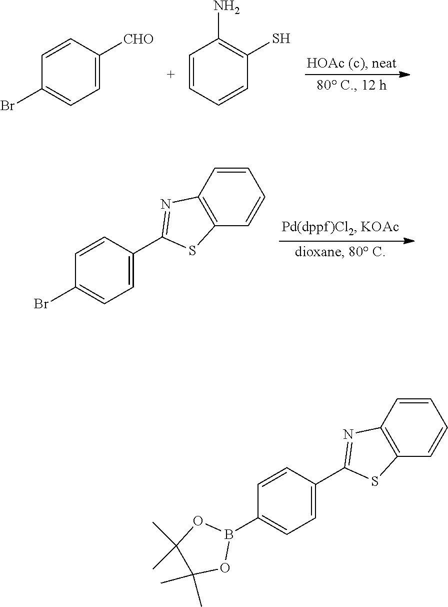 [SMILES: O=Cc1ccc(Br)cc1 ; Nc1ccccc1S ; Brc1ccc(-c2nc3ccccc3s2)cc1 ; CC1(C)OB(c2ccc(-c3nc4ccccc4s3)cc2)OC1(C)C]

### Example (Ex) 1 Synthesis of ETL-1

2,6-Dibromopyridine (1.18 g, 5.0 mmol, 237 g/mol), the boric ester derivative of 2-phenylbenzo[d]thiazole obtained above (3.37 g, 10.0 mmol, 337 g/mol), and Pd(PPh₃)₄ (58 mg, 0.5 mol %, 1154 g/mol) were charged in a 250 mL three-neck flask. 100 mL dry THF and K₂CO₃ (4.55 g, 33 mmol, 138 g/mol, in 16.5 mL DI water) were added into the above mixture under N₂ atmosphere. The mixture was heated to reflux and kept for 8 hours. TLC was utilized to monitor the reaction results. After cooling to room temperature, the resulting compound was filtered and washed with DI water and THF to give white powders. The obtained powders were recrystalized in toluene to give products as white powders with a yield above 90%. ¹H NMR (400 MHz, CDCl₃, ppm): 8.98 (s, 2H), 8.31-8.33 (d, J=8.0 Hz, 4H), 8.55 (s, 1H), 8.12-8.14 (d, J=8.0 Hz, 2H), 7.95-7.97 (d, J=8.0 Hz, 2H), 7.83-7.85 (d, J=8.0 Hz, 4H), 7.52-7.57 (m, 2H), 7.43-7.47 (m, 2H); LC-MS-ESI (m/z): calculated mass for C₃₁H₁₉N₃S₂ 497.10, found (M+H)⁺498.1057; and HPLC purity: 99.8%.

The obtained compound, ETL-1, has the following structure:

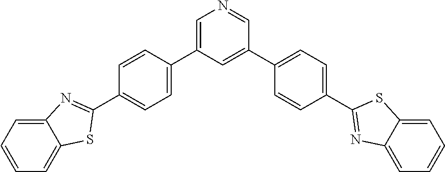 [SMILES: c1ccc2sc(-c3ccc(-c4cncc(-c5ccc(-c6nc7ccccc7s6)cc5)c4)cc3)nc2c1]

ETL-1 obtained above has a HOMO level of −5.97 eV, a LUMO level of −1.94 eV, a triplet energy of 2.42 eV, and an electron mobility level of 0.23, as determined by the modeling method described above.

### Ex 2 Synthesis of ETL-2

3,5-Dibromopyridine (1.18 g, 5.0 mmol, 237 g/mol), the boric ester derivative of 2-phenylbenzo[d]thiazole obtained above (3.37 g, 10.0 mmol, 337 g/mol), and Pd(PPh₃)₄ (58 mg, 0.5 mol %, 1154 g/mol) were charged in a 250 mL three-neck flask. 100 mL dry THF and K₂CO₃ (4.55 g, 33 mmol, 138 g/mol, in 16.5 mL DI water) were added into the above mixture under N₂ atmosphere. The mixture was heated to reflux and kept for 8 hours. TLC was utilized to monitor the reaction results. After cooling to room temperature, the resulting compound was filtered and washed with DI water, THF to give white powders. The obtained powders were recrystalized in toluene to give products as white powders with a yield above 90%. ¹H NMR (400 MHz, CDCl₃, ppm): 8.21-8.34 (m, 8H), 8.10-8.13 (m, 2H), 7.90-7.96 (m, 3H), 7.80-7.84 (m, 2H), 7.51-7.55 (m, 2H), 7.40-7.44 (m, 2H); LC-MS-ESI (m/z): calculated mass for C₃₁H₁₉N₃S₂ 497.10, found (M+H)⁺498.1079; and HPLC purity: 99.4%.

The obtained compound, ETL-2, has the following structure:

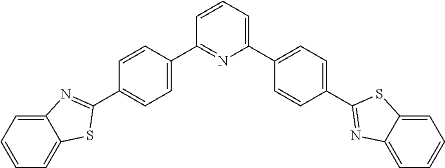 [SMILES: c1cc(-c2ccc(-c3nc4ccccc4s3)cc2)nc(-c2ccc(-c3nc4ccccc4s3)cc2)c1]

ETL-2 obtained above has a HOMO level of −5.81 eV, a LUMO level of −1.93 eV, a triplet energy of 2.36 eV, and an electron mobility level of 0.20, as determined by the modeling method described above.

Thermal properties of ETL-1 and ETL-2 were analyzed by DSC and TGA and results are shown in Table 1. As shown in Table 1, ETL-1 has a T_{d} of 363° C. and a Tₘ of 187° C., and ETL-2 has a T_{d} of 351° C. and a Tₘ of 215° C.

TABLE 1

| Compound | T_{d} (° C.) | T_{g} (° C.) | Tₘ (° C.) |
|---|---|---|---|
| ETL-1 | 363 (onset) | NA* | 187 |
| ETL-2 | 351 (onset) | NA* | 215 |

*No obvious T_{g} was detected by DSC.

### Exs 3-4 and Comp Ex a OLED Device Fabrication

All organic materials were purified by sublimation before deposition. OLEDs were fabricated onto an Indium Tin Oxide (ITO) coated glass substrate that served as the anode, and topped with an aluminum cathode. All organic layers were thermally deposited by chemical vapor deposition, in a vacuum chamber with a base pressure of <10⁻⁷ torr. The deposition rates of organic layers were maintained at 0.1˜0.05 nm/s. The aluminum cathode was deposited at 0.5 nm/s. The active area of the OLED device was “3 mm×3 mm,” as defined by the shadow mask for cathode deposition.

Each cell, containing HIL1, HIL2, HTL, EML host, EML dopant, ETL, or EIL, was placed inside a vacuum chamber, until it reached 10⁻⁶ torr. To evaporate each material, a controlled current was applied to the cell, containing the material, to raise the temperature of the cell. An adequate temperature was applied to keep the evaporation rate of the materials constant throughout the evaporation process.

For the HIL1 layer, N4,N4′-diphenyl-N4,N4′-bis(9-phenyl-9H-carbazol-3-yl)-[1,1′-biphenyl]-4,4′-diamine was evaporated at a constant 1 A/s rate, until the thickness of the layer reached 600 Angstrom. Simultaneously, the dipyrazino[2,3-f:2′,3′-h]quinoxaline-2, 3,6,7,10,11-hexacarbonitrile layer (HTL2 layer) was evaporated at a constant 0.5 A/s rate, until the thickness reached 50 Angstrom. For the HTL layer, N-([1,1′-biphenyl]-4-yl)-9,9-dimethyl-N-(4-(9-phenyl-9H-carbazol-3-yl)phenyl)-9H-fluoren-2-amine was evaporated at a constant 1 A/s rate, until the thickness reached 250 Angstrom. For the EML layer, 9-phenyl-10-(4-phenylnaphthalen-1-yl)anthracene (BH-1, host) and N1,N6-bis(5′-fluoro-[1,1′:3′,1″-terphenyl]-4′-yl)-N1,N6-diphenylpyrene-1,6-diamine (BD-1, dopant) were co-evaporated, until the thickness reached 200 Angstrom. The deposition rate for host material was 0.98 A/s, and the deposition for the dopant material was 0.02 A/s, resulting in a 2% doping of the host material. For the ETL layer, the ETL compounds were co-evaporated with lithium quinolate (Liq), until the thickness reached 300 Angstrom. The evaporation rate for the ETL compounds and Liq was 0.5 A/s. Alq₃ (tris(8-hydroxyquinolinato)aluminum) was used as a reference material to compare with the inventive compounds. Alq₃ was evaporated solely at 1 A/s rate, until 300 Angstrom. Finally, “20 Angstrom” of a thin electron injection layer (Liq) was evaporated at a 0.5 A/s rate. See Table 2.

The current-voltage-brightness (J-V-L) characterizations for the OLED devices were performed with a source measurement unit (KEITHLY 238) and a luminescence meter (MINOLTA CS-100A). Electroluminescence (EL) spectra of the OLED devices were collected by a calibrated CCD spectrograph.

TABLE 2
Device Materials

| | Name |
|---|---|
| Hole Injection | N4,N4′-diphenyl-N4,N4′-bis(9-phenyl-9H- |
| Material (HIL1 layer) | carbazol-3-yl)-[1,1′-biphenyl]-4,4′-diamine |
| Hole Injection | dipyrazino[2,3-f:2′,3′-h]quinoxaline-2,3,6,7,10,11- |
| Material (HIL2 layer) | hexacarbonitrile |
| Hole Transport | N-([1,1′-biphenyl]-4-yl)-9,9-dimethyl-N-(4- |
| Material (HTL layer) | (9-phenyl-9H-carbazol-3-yl)phenyl)-9H-fluoren- |
| | 2-amine |
| Fl Blue Host | 9-phenyl-10-(4-phenylnaphthalen-1-yl)anthracene |
| | (BH-1) |
| Fl Blue Dopant | N1,N6-bis(5′-fluoro-[1,1′:3′,1″-terphenyl]-4′-yl)- |
| | N1,N6-diphenylpyrene-1,6-diamine (BD-1) |
| Electron Transport | Ref ETL: tris(8-hydroxyquinolinato)aluminum |
| Material | (Comp Ex A) |
| | ETL-1:Liq (1:1 weight ratio) (Ex 3) |
| | ETL-2:Liq (1:1 weight ratio) (Ex 4) |
| Electron Injection | lithium quinolate |
| Material | |

Ref ETL, BH-1 and BD-1 described above have the following structure,

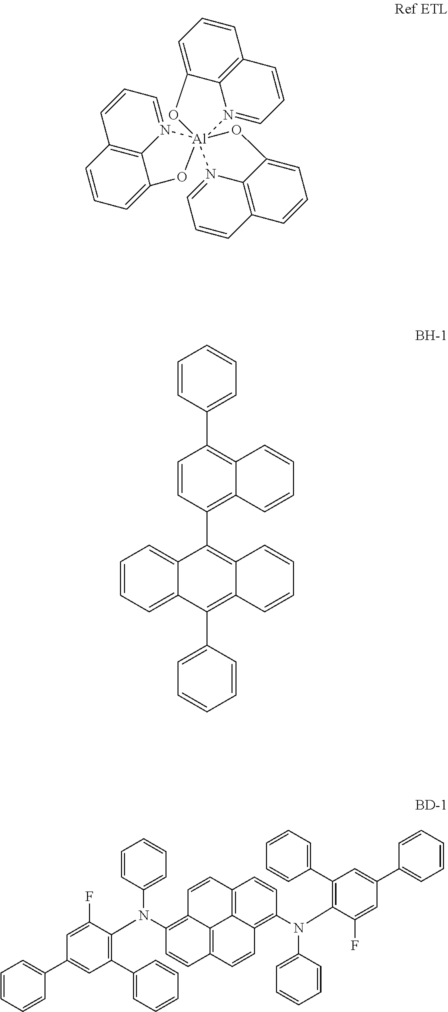

As shown in Table 3, the inventive device containing an ETL film layer containing ETL-1 and ETL-2 showed lower driving voltage, higher efficiency and comparable luminance property, as compared to the OLED device containing the Ref ETL material (Comp Ex A).

TABLE 3

| | | | Luminous | |
|---|---|---|---|---|
| | Electron | Voltage | Efficiency | |
| | Transport | @500 | @500 nit [lm/W] | CIE* |
| Device | Material | nit [V] | [lumens per watt] | (X, Y) |
| Comp | Ref ETL | 5.4 | 2.0 | 0.143, 0.101 |
| Ex A | | | | |
| Ex 3 | ETL-1:Liq (1:1) | 4.4 | 2.8 | 0.139, 0.091 |
| Ex 4 | ETL-2:Liq (1:1) | 4.6 | 2.6 | 0.139, 0.093 |

*CIE refers to International Commission on Illumination.

What is claimed is:

1. A composition comprising an organic compound, wherein the organic compound has the structure selected from one of the following formulae (I-1) through (I-7): 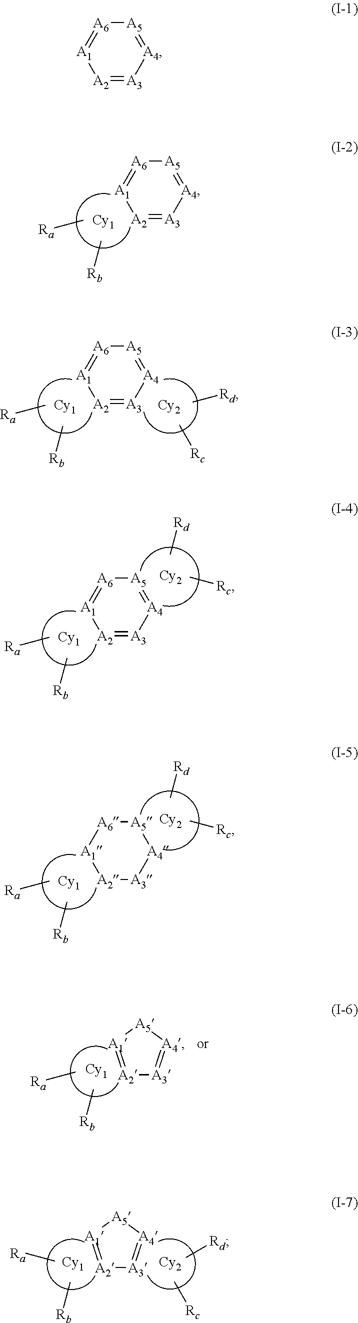
wherein, in formula (I-1), one or two of A₁ through A₆ are each independently selected from N, P, P═O, PR₁R₂ or B; and the remaining A₁ through A₆ are each independently selected from C or CR′; provided that at most one of A₁ through A₆ is N; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; and wherein two of A₁ through A₆ are substituted by Structure A;
wherein, in formula (I-2), A₁ and A₂ are each C; one or two of A₃ through A₆ are each independently selected from N, P, P═O, PR₁R₂ or B; and the remaining A₃ through A₆ are each independently selected from C or CR′; provided that at most one of A₃ through A₆ is N; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ is a five-membered or six-membered ring containing at most two heteroatoms, provided that at most one of the heteroatoms is N; wherein Rₐ and R_{b} are each independently selected from hydrogen, deuterium, a C₁-C₅₀ substituted or unsubstituted alkyl, a substituted or unsubstituted C₁-C₅₀ alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and Rₐ and R_{b} may optionally form a ring; and wherein two of A₃ through A₆ and atoms in Cy₁ are substituted by Structure A;
wherein, in formula (I-3), A₁ through A₄ are each C; one of A₅ and A₆ is selected from N, P, P═O, PR₁R₂ or B; and the remaining A₅ or A₆ is selected from C or CR′; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ and Cy₂ are each independently a five-membered or six-membered ring containing at most two heteroatoms, provided that at most one of the heteroatoms is N; wherein Rₐ, R_{b}, R_{c} and R_{d} are each independently selected from hydrogen, deuterium, a C₁-C₅₀ substituted or unsubstituted alkyl, a substituted or unsubstituted C₁-C₅₀ alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and Rₐ and R_{b}, or R_{c} and R_{d} may optionally form a ring; and wherein two of A₅ through A₆ and atoms in Cy₁ and Cy₂ are substituted by Structure A;
wherein, in formula (I-4), A₁, A₂, A₄ and A₅ are each C; one of A₃ and A₆ is selected from N, P, P═O, PR₁R₂ or B; and the remaining A₃ or A₆ is selected from C or CR′; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ and Cy₂ are each independently a five-membered or six-membered ring containing at most two heteroatoms, provided that at most one of the heteroatoms is N; wherein Rₐ, R_{b}, R_{c} and R_{d} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a substituted or unsubstituted C₆-C₅₀ aryloxy, a substituted or unsubstituted C₆-C₆₀ arylthio, a C₁-C₆₀ substituted or unsubstituted heteroaryl, a halogen or a cyano; and Rₐ and R_{b}, or R_{c} and R_{d} may optionally form a ring; and wherein two of A₃, A₆ and atoms in Cy₁ and Cy₂ are substituted by Structure A;
wherein, in formula (I-5), A₁″, A₂″, A₄″ and A₅″ are each C; and A₃″ and A₆″ are each independently selected from NR′, O, S or CR₁R₂; provided that at least one of A₃″ and A₆″ is selected from NR′, O or S; and at most one of A₃″ and A₆″ is NR′; wherein R′, R₁, and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ and Cy₂ are each independently a five-membered or six-membered ring containing at most two heteroatoms, provided that at most one of the heteroatoms is N; wherein Rₐ, R_{b}, R_{c} and R_{d} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and Rₐ and R_{b} may optionally form a ring; and wherein two of atoms in Cy₁ and Cy₂ are substituted by Structure A;
wherein, in formula (I-6), A₁′ and A₂′ are each C; A₃′ and A₄′ are each independently selected from C, CR′, N, P, P═O, PR₁R₂ or B; and A₅′ is selected from O, S, NR′ or CR₁R₂; provided that only one or two of A₁′ through A₅′ are not C, CR′ or CR₁R₂; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ is a five-membered or six-membered ring containing at most two heteroatoms, provided that at most one of the hertoatoms is N; wherein Rₐ and R_{b} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and Rₐ and R_{b} may optionally form a ring; and wherein two of A₃ through A₄ and atoms in Cy₁ are substituted by Structure A;
wherein, in formula (I-7), A₁′ through A₄′ are each C; and A₅′ is selected from O, S or NR′; wherein R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; wherein Cy₁ and Cy₂ are each independently a five-membered or six-membered ring containing at most two heteroatoms, provided that at most one of the heteroatoms is N; wherein Rₐ, R_{b}, R_{c} and R_{d} are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and Rₐ and R_{b}, or R_{c} and R_{d} may optionally form a ring; and wherein two of atoms in Cy₁ and Cy₂ are substituted by Structure A; and
wherein Structure A has the following structure: 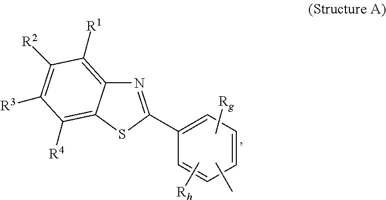
wherein R¹ through R⁴, R_{g} and Rₕ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a substituted or unsubstituted C₆-C₆₀ aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and R_{g} and Rₕ may optionally form a ring.

2. The composition of claim 1, wherein, in each of formulae (I-1), (I-2), (I-3), (I-4), (I-5), (I-6) and (I-7), R¹ through R⁴, R_{g} and Rₕ are each independently selected from hydrogen, a C₆-C₆₀ substituted or unsubstituted aryl, or a C₁-C₆₀ substituted or unsubstituted heteroaryl.

3. The composition of claim 1, wherein, in formula (I-1), one of A₁ through A₆ is N and the remaining A₁ through A₆ are each independently selected from C or CR′;
wherein, in formula (I-2), one of A₃ through A₆ is N, and the remaining A₃ through A₆ are each independently selected from C or CR′;
wherein, in formula (I-3), one of A₅ and A₆ is N, and the remaining A₅ or A₆ is selected from C or CR′;
wherein, in formula (I-4), one of A₃ and A₆ is N, and the remaining A₃ or A₆ is selected from C or CR′;
wherein, in formula (I-5), at least one of A₃″ and A₆″ is NR′;
wherein, in formula (I-6), A₃′ and A₄′ are each independently selected from CR′; and A₅′ is selected from O, S, NR′ or CR₁R₂; and
wherein, in formula (I-7), A₁′ through A₄′ are each C, and A₅′ is O or S.

4. The composition of claim 1, wherein the organic compound of formula (I-2) has the structure represented by formula (II-2a) or (II-2b): 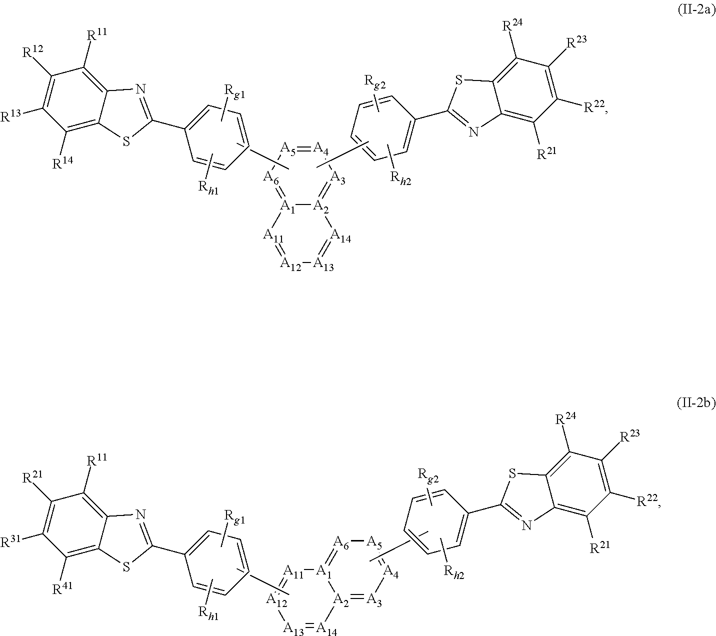
wherein, in each of formulae (II-2a) and (II-2b), A₁ and A₂ are each C; and A₃ through A₆ and A₁₁ through A₁₄ are each independently selected from CR′, N, P, P═O, PR₁R₂ or B; provided that one of A₃ through A₆ is N and at most one of A₁₁ through A₁₄ is N;
wherein, in each of formulae (II-2a) and (II-2b), R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; and
wherein, in each of formulae (II-2a) and (II-2b), R¹¹ through R¹⁴, R²¹ through R²⁴, R_{g1}, Rₕ₁, R_{g1} and Rₕ₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen, or a cyano; and R_{g1} and Rₕ₁, R_{g2} and Rₕ₂ may optionally form a ring, and two adjacent of R¹¹ through R¹⁴ or R²¹ through R²⁴ may optionally form a ring.

5. The composition of claim 1, wherein the organic compound of formula (I-3) has the structure represented by formula (II-3a) or (II-3b): 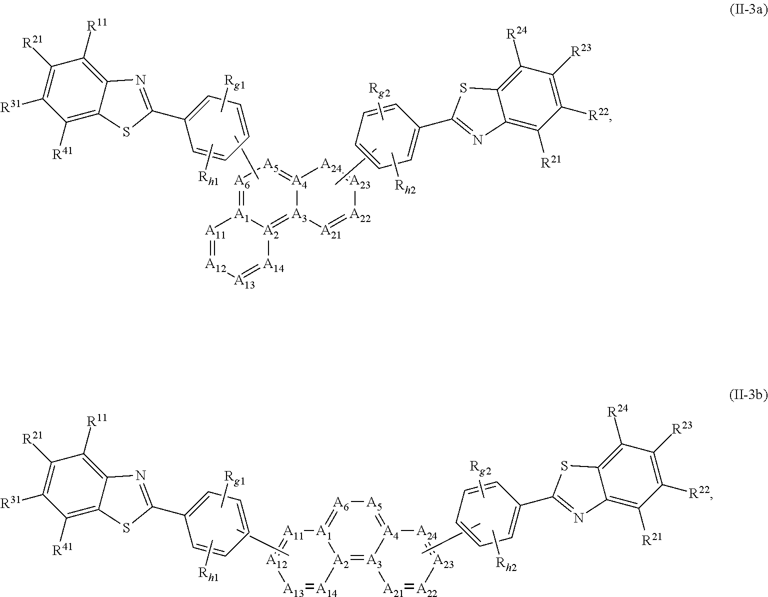
wherein, in each of formulae (II-3a) and (II-3b), A₁ through A₄ are each C; one of A₅ and A₆ is selected from N, P, P═O, PR₁R₂ or B; and the remaining A₅ or A₆ is selected from C or CR′; and A₁₁ through A₁₄ and A₂₁ through A₂₄ are each independently selected from C, CR′, N, P, P═O, PR₁R₂ or B; provided that at most one of A₁₁ through A₁₄ is N, and at most one of A₂₁ through A₂₄ is N;
wherein, in each of formulae (II-3a) and (II-3b), R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; and
wherein, in each of formulae (II-3a) and (II-3b), R¹¹ through R¹⁴, R²¹ through R²⁴, R_{g1}, Rₕ₁, R_{g1} and Rₕ₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen, or a cyano; and R_{g1} and Rₕ₁, R_{g2} and Rₕ₂ may optionally form a ring, and two adjacent of R¹¹ through R¹⁴ or R²¹ through R²⁴ may optionally form a ring.

6. The composition of claim 1, wherein the organic compound of formula (I-4) has the structure represented by formula (II-4): 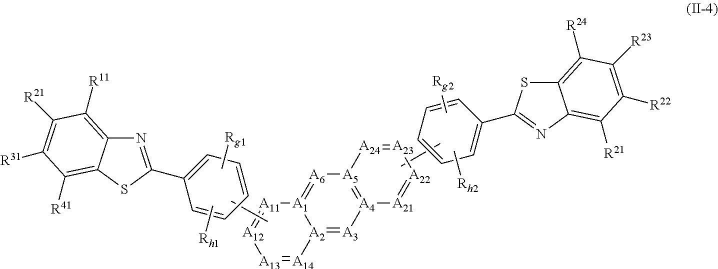
wherein, in formula (II-4), A₁, A₂, A₄ and A₅ are each C; one of A₃ and A₆ is selected from N, P, P═O, PR₁R₂ or B; and the remaining A₃ or A₆ is selected from C or CR′; and A₁₁ through A₁₄, and A₂₁ through A₂₄ are each independently selected from C, CR′, N, P, P═O, PR₁R₂ or B; provided that at most one of A₁₁ through A₁₄ is N, and at most one of A₂₁ through A₂₄ is N;
wherein, in formula (II-4), R′, R₁, and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; and
wherein, in formula (II-4), R¹¹ through R¹⁴, R²¹ through R²⁴, R_{g1}, Rₕ₁, R_{g1} and Rₕ₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen, or a cyano; and R_{g1} and Rₕ₁, R_{g2} and Rₕ₂ may optionally form a ring, and two adjacent of R¹¹ through R¹⁴ or R²¹ through R²⁴ may optionally form a ring.

7. The composition of claim 1, wherein the organic compound of formula (I-5) has the structure represented by formula (II-5): 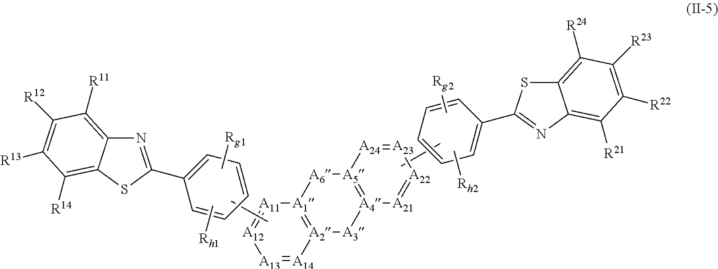
wherein, in formula (II-5), A₁″, A₂″, A₄″ and A₅″ are each C; A₃″ and A₆″ are each independently selected from NR′, O, S, or CR₁R₂; A₁ through A₁₄ and A₂₁ through A₂₄ are each independently selected from C, CR′, N, P, P═O, PR₁R₂, or B; provided that one of A₃″ and A₆″ is NR′, at most one of A₁ through A₁₄ is N, and at most one of A₂₁ through A₂₄ is N;
wherein, in formula (II-5), R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a substituted or unsubstituted C₆-C₅₀ aryloxy, a C₆-C₆₀ substituted or unsubstituted arylthio, or a C₁-C₆₀ substituted or unsubstituted heteroaryl; and
wherein, in formula (II-5), R¹¹ through R¹⁴, R²¹ through R²⁴, R_{g1}, Rₕ₁, R_{g1} and Rₕ₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and R_{g1} and Rₕ₁, R_{g2} and Rₕ₂ may optionally form a ring, and two adjacent of R¹¹ through R¹⁴ or R²¹ through R²⁴ may optionally form a ring.

8. The composition of claim 1, wherein the organic compound of formula (I-6) has the structure represented by formula (II-6a) or (II-6b): 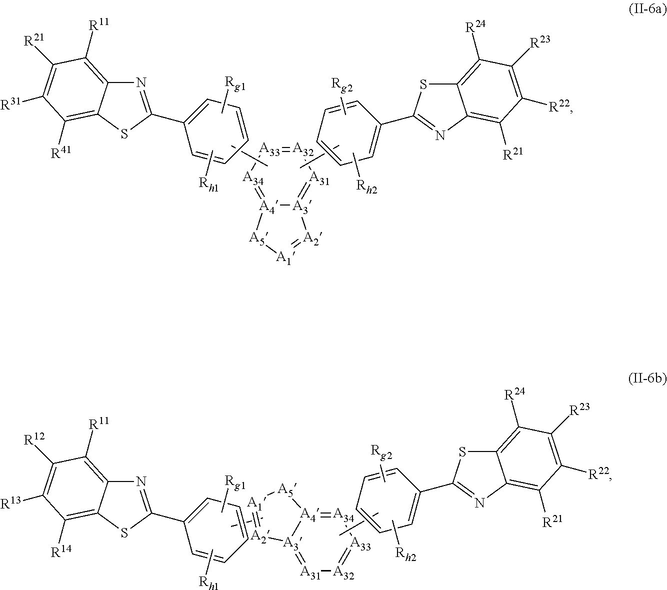
wherein, in each of formulae (II-6a) and (II-6b), A₃′ and A₄′ are each C; A₁′ and A₂′ are each independently selected from C, CR′, N, P, P═O, PR₁R₂ or B; A₅′ is selected from O, S, NR′ or CR₁R₂; and A₃₁ through A₃₄ are each independently selected from C, CR′, N, P, P═O, PR₁R₂ or B; provided that only one or two of A₁′, A₂′ and A₅′ are not C, CR′ or CR₁R₂; and at most one of A₃₁ through A₃₃ is N;
wherein, in each of formulae (II-6a) and (II-6b), R′, R₁, and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; and
wherein, in each of formulae (II-6a) and (II-6b), R¹¹ through R¹⁴, R²¹ through R²⁴, R_{g1}, Rₕ₁, R_{g1} and Rₕ₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen, or a cyano; and R_{g1} and Rₕ₁, R_{g2} and Rₕ₂ may optionally form a ring, and two adjacent of R¹¹ through R¹⁴ or R²¹ through R²⁴ may optionally form a ring.

9. The composition of claim 1, wherein the organic compound of formula (I-7) has the structure represented by formula (II-7): 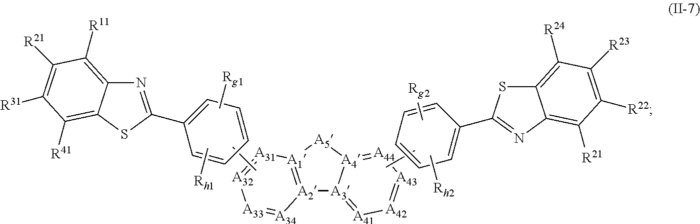
wherein, in formula (II-7), A₁′ through A₄′ are each C; A₅′ is selected from O, S, or NR′; and A₃₁ through A₃₄ and A₄₁ through A₄₄ are each independently selected from C, CR′, N, P, P═O, PR₁R₂ or B; provided that at most one of A₃₁ through A₃₃ is N, and at most one of A₄₁ through A₄₄ is N;
wherein, in formula (II-7), R′, R₁ and R₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, or a C₁-C₆₀ substituted or unsubstituted heteroaryl; and
wherein, in formula (II-7), R¹¹ through R¹⁴, R²¹ through R²⁴, R_{g1}, Rₕ₁, R_{g1} and Rₕ₂ are each independently selected from hydrogen, deuterium, a C₁-C₅₀ substituted or unsubstituted alkyl, a C₁-C₅₀ substituted or unsubstituted alkoxy, a C₁-C₅₀ substituted or unsubstituted alkoxycarbonyl, a C₆-C₆₀ substituted or unsubstituted aryl, a C₆-C₅₀ substituted or unsubstituted aryloxy, a C₆-C₆₀ substituted or unsubstituted arylthio, a C₁-C₆₀ substituted or unsubstituted heteroaryl, a halogen or a cyano; and R_{g1} and Rₕ₁, R_{g2} and Rₕ₂ may optionally form a ring, and two adjacent of R¹¹ through R¹⁴ or R²¹ through R²⁴ may optionally form a ring.

10. The composition of any one of claims 4-9, wherein R¹¹ through R¹⁴, R²¹ through R²⁴, R_{g1}, Rₕ₁, R_{g2}, and Rₕ₂ are each independently selected from hydrogen, a C₆-C₆₀ substituted or unsubstitutedaryl, or a C₁-C₆₀ substituted or unsubstituted heteroaryl.

11. The composition of claim 1, wherein the organic compound is selected from one of the following compounds (1) through (26):

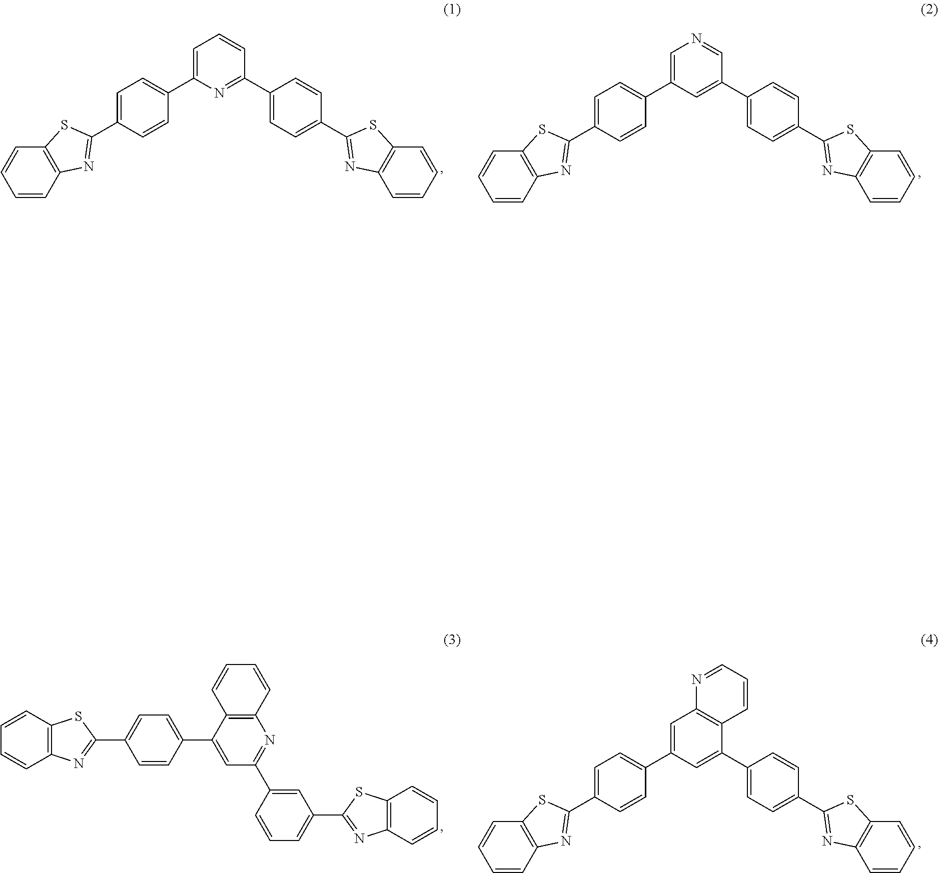 [SMILES: c1cc(-c2ccc(-c3nc4ccccc4s3)cc2)nc(-c2ccc(-c3nc4ccccc4s3)cc2)c1 ; c1ccc2sc(-c3ccc(-c4cncc(-c5ccc(-c6nc7ccccc7s6)cc5)c4)cc3)nc2c1 ; c1cc(-c2cc(-c3ccc(-c4nc5ccccc5s4)cc3)c3ccccc3n2)cc(-c2nc3ccccc3s2)c1 ; c1ccc2sc(-c3ccc(-c4cc(-c5ccc(-c6nc7ccccc7s6)cc5)c5cccnc5c4)cc3)nc2c1]

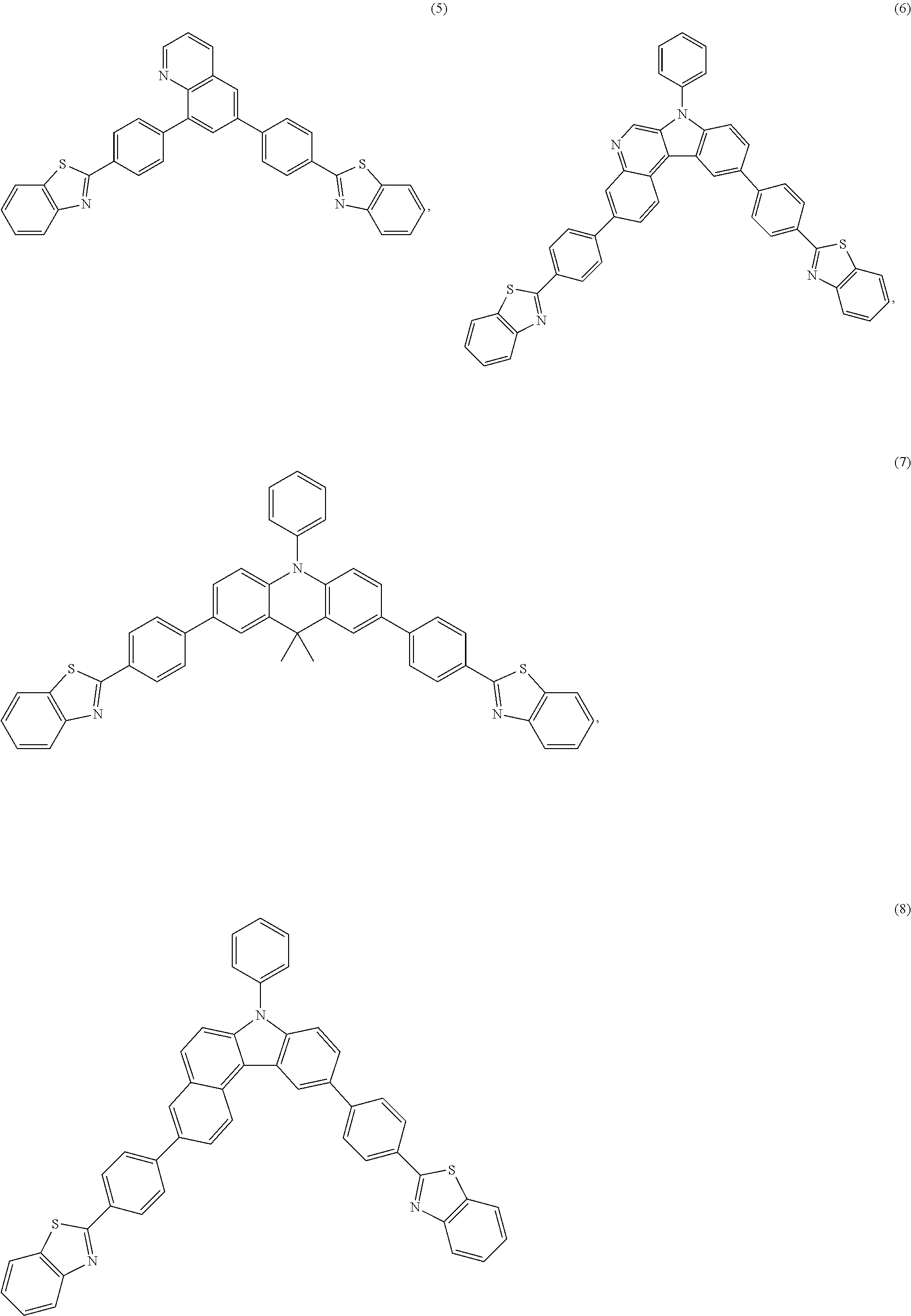 [SMILES: c1cnc2c(-c3ccc(-c4nc5ccccc5s4)cc3)cc(-c3ccc(-c4nc5ccccc5s4)cc3)cc2c1 ; c1ccc(-n2c3ccc(-c4ccc(-c5nc6ccccc6s5)cc4)cc3c3c4ccc(-c5ccc(-c6nc7ccccc7s6)cc5)cc4ncc32)cc1 ; CC1(C)c2cc(-c3ccc(-c4nc5ccccc5s4)cc3)ccc2N(c2ccccc2)c2ccc(-c3ccc(-c4nc5ccccc5s4)cc3)cc21 ; c1ccc(-n2c3ccc(-c4ccc(-c5nc6ccccc6s5)cc4)cc3c3c4ccc(-c5ccc(-c6nc7ccccc7s6)cc5)cc4ccc32)cc1]

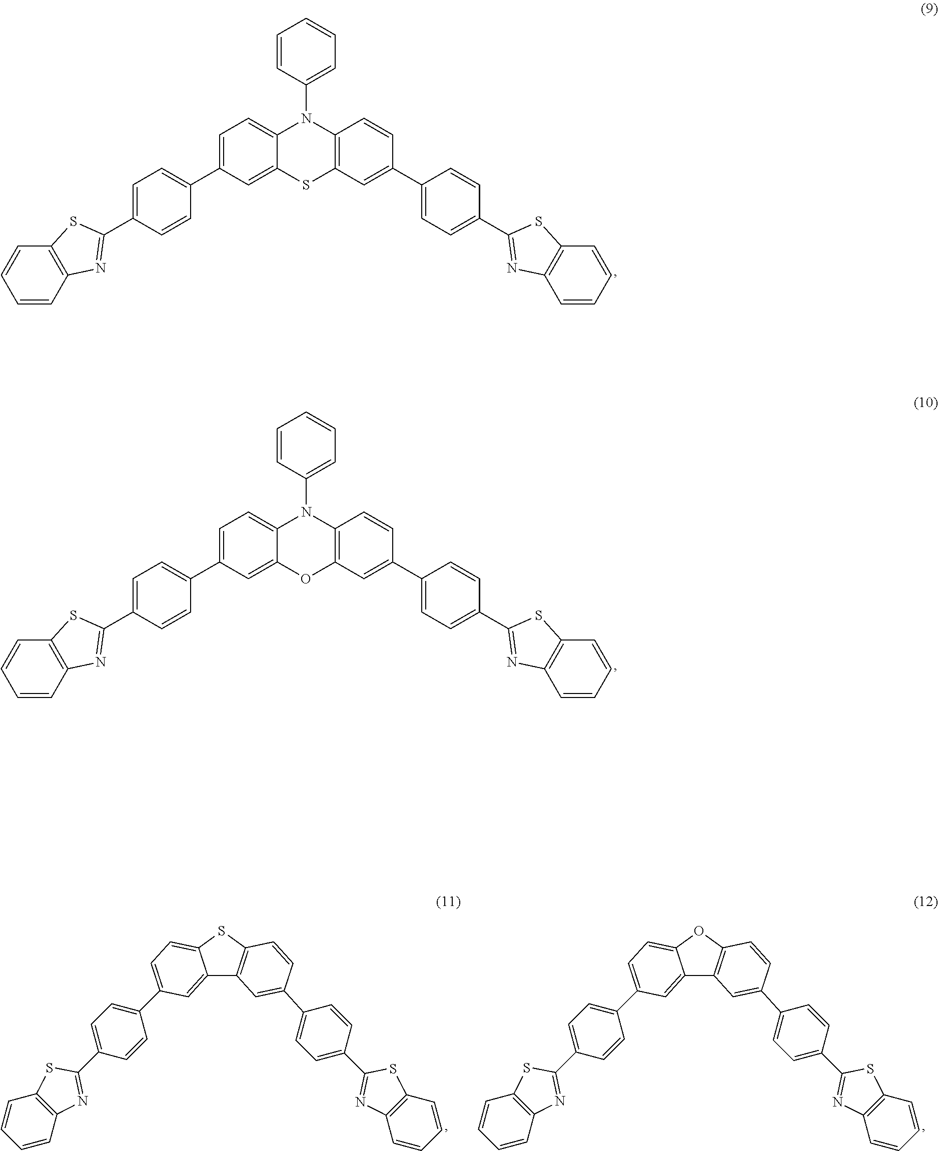

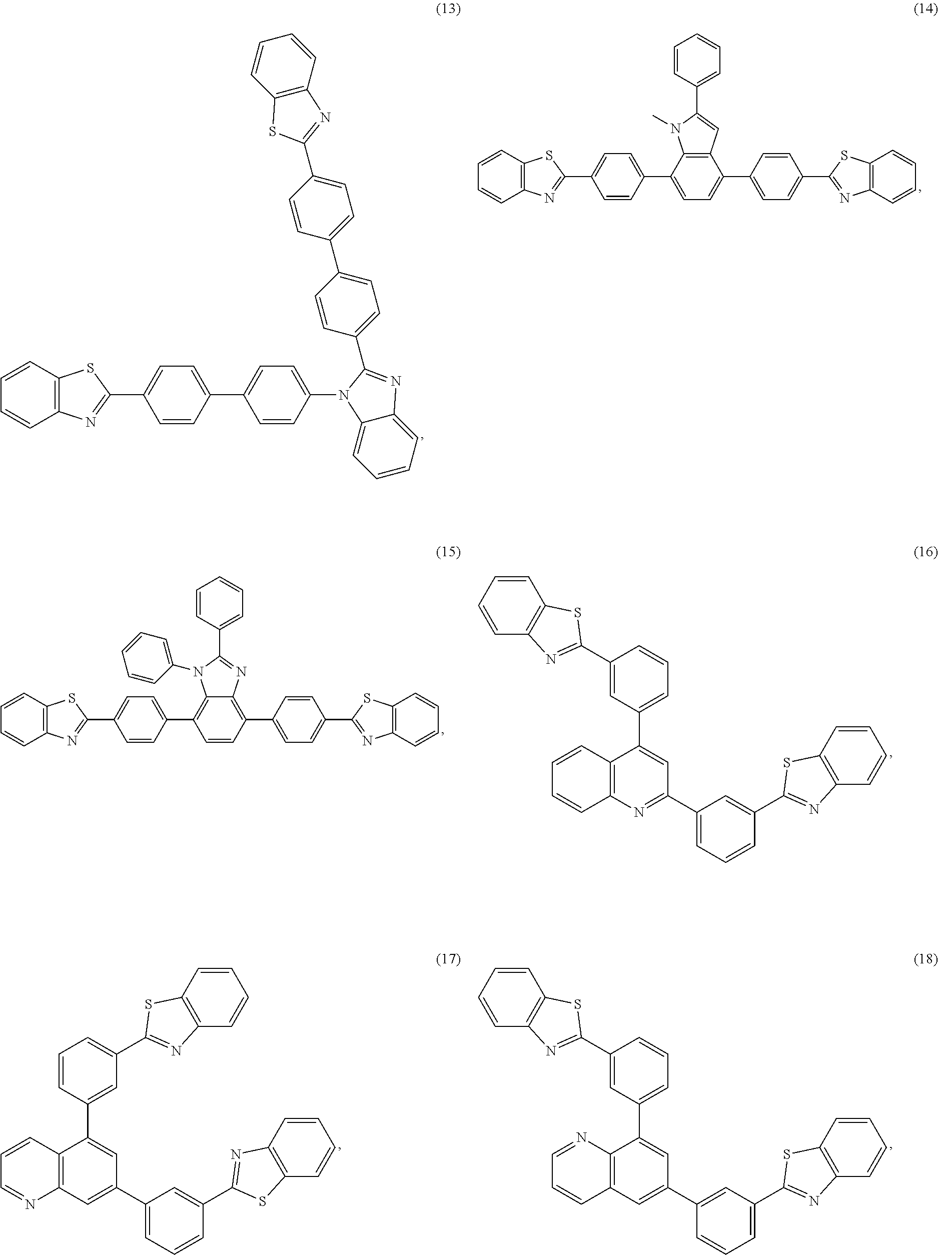 [SMILES: c1ccc2sc(-c3ccc(-c4ccc(-c5nc6ccccc6n5-c5ccc(-c6ccc(-c7nc8ccccc8s7)cc6)cc5)cc4)cc3)nc2c1 ; Cn1c(-c2ccccc2)cc2c(-c3ccc(-c4nc5ccccc5s4)cc3)ccc(-c3ccc(-c4nc5ccccc5s4)cc3)c21 ; c1ccc(-c2nc3c(-c4ccc(-c5nc6ccccc6s5)cc4)ccc(-c4ccc(-c5nc6ccccc6s5)cc4)c3n2-c2ccccc2)cc1 ; c1cc(-c2cc(-c3cccc(-c4nc5ccccc5s4)c3)c3ccccc3n2)cc(-c2nc3ccccc3s2)c1 ; c1cc(-c2cc(-c3cccc(-c4nc5ccccc5s4)c3)c3cccnc3c2)cc(-c2nc3ccccc3s2)c1 ; c1cc(-c2cc(-c3cccc(-c4nc5ccccc5s4)c3)c3ncccc3c2)cc(-c2nc3ccccc3s2)c1]

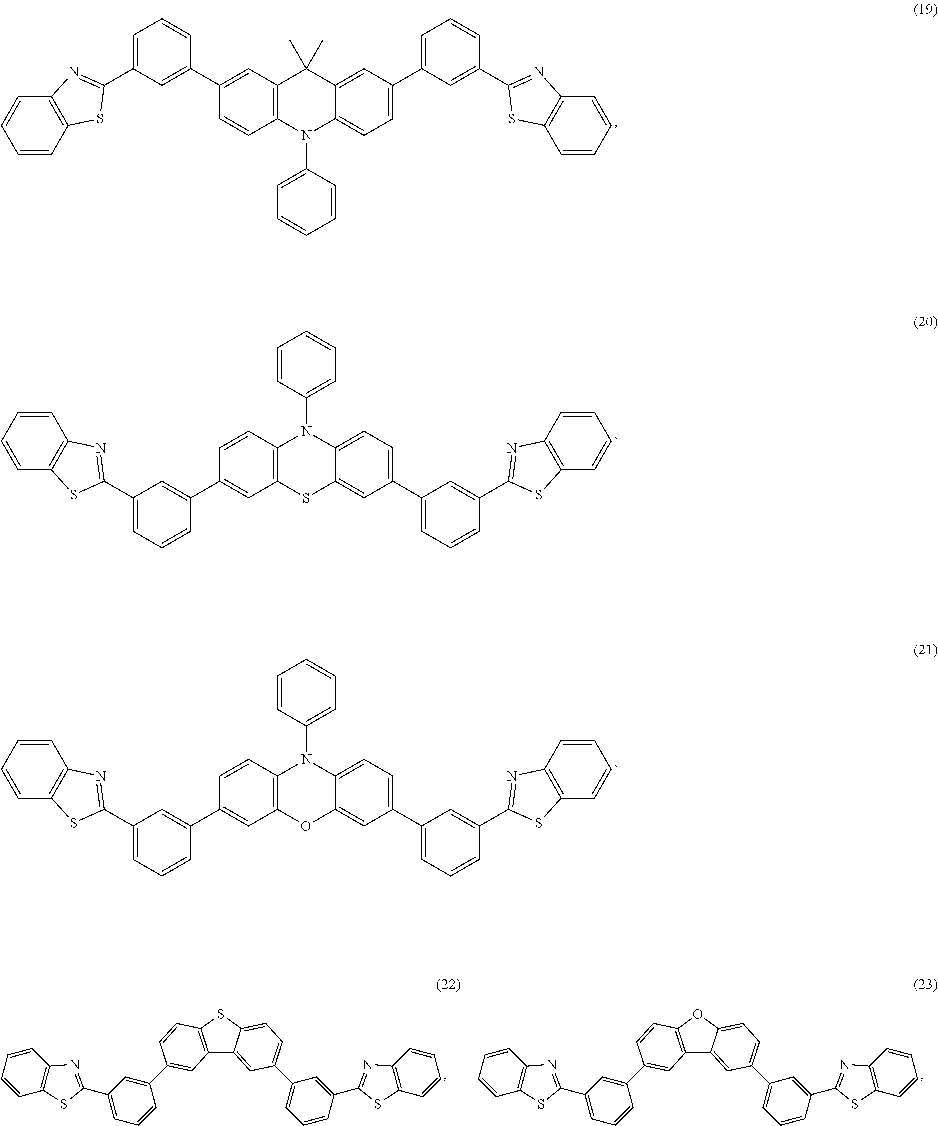

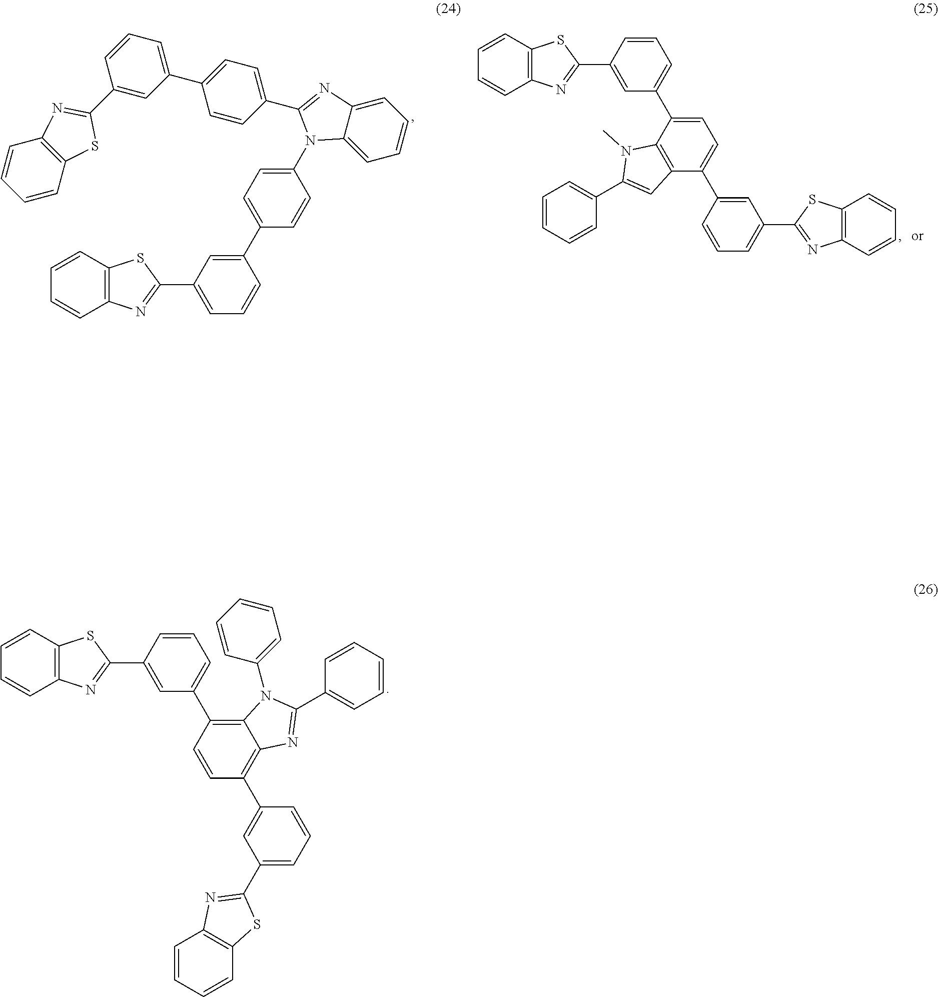 [SMILES: c1cc(-c2ccc(-c3nc4ccccc4n3-c3ccc(-c4cccc(-c5nc6ccccc6s5)c4)cc3)cc2)cc(-c2nc3ccccc3s2)c1 ; Cn1c(-c2ccccc2)cc2c(-c3cccc(-c4nc5ccccc5s4)c3)ccc(-c3cccc(-c4nc5ccccc5s4)c3)c21 ; c1ccc(-c2nc3c(-c4cccc(-c5nc6ccccc6s5)c4)ccc(-c4cccc(-c5nc6ccccc6s5)c4)c3n2-c2ccccc2)cc1]

12. The composition of claim 1 further comprising a dopant.

13. An electronic device comprising an organic layer, wherein the organic layer comprises the composition of any one of claims 1-12.

14. The electronic device of claim 13, wherein the organic layer comprises a hole transport layer, an emissive layer, an electron transport layer, or a hole injection layer.

15. The electronic device of claim 14, wherein the electronic device is a light emitting device.
